SEQUENCE LISTING (20 sequences)

SEQ ID NO: 1 - protein, 5 aa, Artificial Sequence
  HCDR1, EGFR binding arm
TYGMH

SEQ ID NO: 2 - protein, 17 aa, Artificial Sequence
  HCDR2, EGFR binding arm
VIWDDGSYKYYGDSVKG

SEQ ID NO: 3 - protein, 16 aa, Artificial Sequence
  HCDR3, EGFR binding arm
DGITMVRGVMKDYFDY

SEQ ID NO: 4 - protein, 11 aa, Artificial Sequence
  LCDR1, EGFR binding arm
RASQDISSALV

SEQ ID NO: 5 - protein, 7 aa, Artificial Sequence
  LCDR2, EGFR binding arm
DASSLES

SEQ ID NO: 6 - protein, 9 aa, Artificial Sequence
  LCDR3, EGFR binding arm
QQFNSYPLT

SEQ ID NO: 7 - protein, 5 aa, Artificial Sequence
  HCDR1, c-Met binding arm
SYGIS

SEQ ID NO: 8 - protein, 17 aa, Artificial Sequence
  HCDR2, c-Met binding arm
WISAYNGYTNYAQKLQG

SEQ ID NO: 9 - protein, 10 aa, Artificial Sequence
  HCDR3, c-Met binding arm
DLRGTNYFDY

SEQ ID NO: 10 - protein, 11 aa, Artificial Sequence
  LCDR1, c-Met binding arm
RASQGISNWLA

SEQ ID NO: 11 - protein, 7 aa, Artificial Sequence
  LCDR2, c-Met binding arm
AASSLLS

SEQ ID NO: 12 - protein, 9 aa, Artificial Sequence
  LCDR3, c-Met binding arm
QQANSFPIT

SEQ ID NO: 13 - protein, 125 aa, Artificial Sequence [sequence omitted]

SEQ ID NO: 14 - protein, 107 aa, Artificial Sequence [sequence omitted]

SEQ ID NO: 15 - protein, 119 aa, Artificial Sequence
  VH, c-Met binding arm
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Artificial Sequence
  VL, c-Met binding arm
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Artificial Sequence
  Heavy chain 1
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Artificial Sequence
  Light chain 1
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Artificial Sequence
  Heavy chain 2
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Artificial Sequence
  Light chain 2
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0372581 A1
(43) Pub. Date: Nov. 24, 2022

(54) Methods for Identifying Cancer Patients for Combination Treatment

(71) Applicant: Janssen Biotech, Inc., Horsham, PA (US)

(72) Inventor: Joshua Curtin, Jenkintown, PA (US)

(21) Appl. No.: 17/747,346

(22) Filed: May 18, 2022

Related U.S. Application Data
(60) Provisional application No. 63/190,004, filed on May 18, 2021.

(51) Int. Cl. C12Q 1/6886; C07K 16/28; A61K 39/395; A61K 31/5377; G01N 33/574; A61P 35/00

(52) U.S. Cl. CPC C12Q 1/6886; C07K 16/2863; A61K 39/39558; A61K 31/5377; G01N 33/57492; G01N 33/57423; A61P 35/00; C12Q 2600/156; C12Q 2600/106; C07K 2317/31; C07K 2317/51; C07K 2317/515; G01N 2333/71; A61K 2039/505

(57) ABSTRACT

The present disclosure provides methods and kits for determining whether a cancer in a subject is susceptible to a treatment with a combination therapy comprising a bispecific anti-epidermal growth factor receptor (EGFR)/hepatocyte growth factor receptor (c-Met) antibody and an EGFR tyrosine kinase inhibitor (TKI). The present disclosure also provides methods for treating a cancer in a subject based on the susceptibility of the cancer to the treatment with a combination therapy comprising a bispecific EGFR/c-Met antibody and an EGFR TKI.

### CROSS-REFERENCE TO RELATED APPLICATIONS

This application claims the benefit of U.S. Provisional Patent Application No. 63/190,004, filed May 18, 2021, the disclosure of which is herein incorporated by reference in its entirety.

### SEQUENCE LISTING

The sequence listing of the present application is submitted electronically as an ASCII formatted sequence listing with a file name “JBI6555USNP1SEQLIST.TXT”, creation date of May 17, 2022, and a size of 19 kilobytes (KB). This sequence listing submitted is part of the specification and is herein incorporated by reference in its entirety.

### FIELD

The present disclosure relates to methods and kits for identifying and treating cancer patients who would benefit from treatment with a combination therapy comprising a bispecific anti-epidermal growth factor receptor (EGFR)/hepatocyte growth factor receptor (c-Met) antibody and an EGFR tyrosine kinase inhibitor (TKI).

### BACKGROUND

The individual roles of both epidermal growth factor receptor (EGFR) and receptor tyrosine kinase mesenchymal-epithelial transition factor (c-Met) in cancer is well established, making these targets attractive for combination therapy. Both receptors signal through the same survival and anti-apoptotic pathways (ERK and AKT); thus, inhibiting the pair in combination may limit the potential for compensatory pathway activation thereby improving overall efficacy.

Molecular segmentation of advanced non-small cell lung cancer (NSCLC) based on oncogenic driver mutations has improved the overall survival and quality of life for patients with actionable driver mutations and solidified solid tumor targeted therapy. In NSCLC, specific mutations in the EGFR gene are associated with high response rates to EGFR tyrosine kinase inhibitors (EGFR-TKIs). Although the majority of NSCLC patients with EGFR mutations initially respond to EGFR TKI therapy, virtually all acquire resistance that prevents a durable response. Nearly 60% of all tumors that become resistant to EGFR tyrosine kinase inhibitors increase c-Met expression, amplify the c-Met gene, or increase its only known ligand, Hepatocyte Growth Factor (Turke et al., Cancer Cell, 17:77-88, 2010).

Progression of acquired resistance to EGFR-TKI such as osimertinib in epidermal growth factor receptor mutant (EGFRm) NSCLC likely arises from complex and heterogenous patterns of resistance together with co-occurrence of multiple resistance mechanisms, and as such the details of such mechanisms remain elusive.

Amivantamab is a fully human bispecific antibody that targets both EGFR and receptor tyrosine kinase mesenchymal-epithelial transition factor (c-Met), and comprises a fragment crystallizable (Fc) region that has been shown to exhibit immune cell-directing activity (Vijayaraghavan et al., Mol Cancer Ther 19:2044, 2020). Aminvantamab demonstrates clinical activity across diverse EGFRm NSCLC, and was granted breakthrough therapy designation for EGFRm exon 20 insertion NSCLC post-chemotherapy in the United States and China (Haura et al., JCO 37:9009, 2019; Park et al., JCO 38:9512, 2020; Sabari et al., JTO 16:S108, 2021).

Lazertinib is a potential third generation tyrosine kinase inhibitor (TKI) with efficacy in activating EGFR mutations T790M, and central nervous system (CNS) disease (Ahn et al., Lancet Oncol 20:P1681, 2019; Kim et al., JCO 38:9571, 2020). Lazertinib is associated with low rates of EGFR-related toxicity, e.g., rash and diarrehhea, as well as low cardiovascular risk, and hence possesses a safety profile that supports its combination with other anti-EGFR molecules (Ahn et al., Lancet Oncol 20:P1681, 2019; Haddish-Berhane et al., JTO 16:S677, 2022).

### SUMMARY

As specified in the Background section above, there is a need in the art for identifying and treating cancer patients who would benefit from treatment with a combination therapy comprising a bispecific anti-epidermal growth factor receptor (EGFR)/hepatocyte growth factor receptor (c-Met) antibody and an EGFR tyrosine kinase inhibitor (TKI).

In one aspect, provided herein is a method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising
a) determining the presence of one or more mutations in tumor DNA obtained from the subject, wherein the one or more mutations are selected from mutations in one or more genes from the RAS/RAF/MEK pathway and mutations in PIK3CA; and
b) (i) identifying the cancer in the subject as susceptible to the treatment with the combination therapy, when tumor DNA from said subject has no said mutations, or (ii) identifying the cancer in the subject as not susceptible to the treatment with the combination therapy, when tumor DNA from said subject has one or more said mutations.

In one aspect, provided herein is a method for treating a cancer in a subject in need thereof, said method comprising
a) determining the presence of one or more mutations in tumor DNA obtained from the subject, wherein the one or more mutations are selected from mutations in one or more genes from the RAS/RAF/MEK pathway and mutations in PIK3CA; and
b) (i) when tumor DNA from said subject has no said mutations, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI), or (ii) when tumor DNA from said subject has one or more said mutations, administering to the subject a cancer therapy which does not include the combination therapy used in (i).

In some embodiments of the diagnostic or treatment method described above, the one or more genes from RAS/RAF/MEK pathway are FGFR3, KRAS, BRAF, ERBB2, ALK, NRAS, PDGFRA and/or RET. In some embodiments, the mutations in one or more genes from RAS/RAF/MEK pathway comprises FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions. In some embodiments, the KRAS G12X mutations are KRAS G12D, KRAS G12A, KRAS G12C, and KRAS G12V.

In some embodiments of the diagnostic or treatment method described above, wherein the mutations in PIK3CA comprise PIK3CA E545K.

In some embodiments of the diagnostic or treatment method described above, the one or more mutations are further selected from mutations in one or more genes from the WNT/b-catenin pathway. In some embodiments, the one or more genes from WNT/b-catenin pathway are APC and CTNNB1. In some embodiments, the mutations in one or more genes from WNT/b-catenin pathway comprise APC Q1469, APC R405, APC S713, CTNNB1 S33P, CTNNB1 S37C, CTNNB1 S37F, and CTNNB1 S45P.

In another aspect, provided herein is a method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising
a) determining the presence of one or more mutations in tumor DNA obtained from the subject, wherein the one or more mutations are selected from the following two groups:
   (1) PIK3CA E545K, PIK3CA E542K/V, PIK3CA H1047R, PIK3CA amplification, KRAS G12V/C/D/X (X being any amino acid other than G, V, C and D), KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletions, PTEN N48K, CDKN2A G101W, CDKN2B mutations, ALK fusions, FGFR3-TACC3 fusion, TPM3-NTRK1 fusion, RET fusions, BRAF fusions, and other oncogenic fusion events;
   (2) EGFR C797S, EGFR L792H, EGFR amplification, EGFR G796S, EGFR L718X (X being any amino acid), EGFR E709K, EGFR G724S, MET amplification, and MET exon 14 skipping (METex14) mutations; and
b) (i) identifying the cancer in the subject as susceptible to the treatment with the combination therapy, when tumor DNA from said subject has either no mutations from group (1), or has one or more mutations from group (1) and one or more mutations from group (2), or (ii) identifying the cancer in the subject as not susceptible to the treatment with the combination therapy, when tumor DNA from said subject has one or more mutations from group (1) and no mutations from group (2).

In another aspect, provided herein is a method for treating a cancer in a subject in need thereof, said method comprising
a) determining the presence of one or more mutations in tumor DNA obtained from the subject, wherein the one or more mutations are selected from the following two groups:
   (1) PIK3CA E545K, PIK3CA E542K/V, PIK3CA H1047R, PIK3CA amplification, KRAS G12V/C/D/X, KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletion, PTEN N48K, CDKN2A G101W, CDKN2B, ALK fusions, FGFR3-TACC3 and other fusions, RET fusions, BRAF fusions, and other oncogenic fusion events;
   (2) EGFR C797S, EGFR L792H, EGFR amplification, EGFR G796S, EGFR L718X (X being any amino acid), EGFR E709K, EGFR G724S, MET amplification, and MET exon 14 skipping (METex14) mutations; and
b) (i) when tumor DNA from said subject has either no mutations from group (1), or has one or more mutations from group (1) and one or more mutations from group (2), administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI), or (ii) when tumor DNA from said subject has one or more mutations from group (1) and no mutations from group (2), administering to the subject a cancer therapy which does not include the combination therapy used in (i).

In some embodiments of the diagnostic or treatment method described above, the HER2 oncogenic alterations comprise HER2 Y772_A775 duplication, HER2 L755M/S/W and HER2 S310F/Y. In some embodiments, the PTEN deletions comprise PTEN I33del and PTEN I14del. In some embodiments, the ALK fusions comprise SQSTM1-ALK fusion and EML4-ALK fusion. In some embodiments, the RET fusions comprise CCDC6-RET fusion, KIF5B-RET fusion, and NCOA4-RET fusion.

In some embodiments of the diagnostic or treatment method described above, the cancer is a lung cancer, such as a non-small cell lung cancer (NSCLC).

In some embodiments of the diagnostic or treatment method described above, the cancer in the subject is resistant to treatment with an EGFR TKI which is not the same as the EGFR TKI used in the combination therapy. In some embodiments, the EGFR TKI to which the cancer is resistant is selected from osimertinib, erlotinib, afatinib, rociletinib, olmutinib, and any combination thereof. In one embodiment, the EGFR TKI to which the cancer is resistant is osimertinib.

In some embodiments of the diagnostic or treatment method described above, the subject is chemotherapy naïve.

In some embodiments of the diagnostic or treatment method described above, the tumor DNA from the subject has at least one EGFR-activating mutation. In some embodiments the EGFR-activating mutation is selected from exon 19 deletions, L858R, and T790M.

In some embodiments of the diagnostic or treatment method described above, the tumor DNA is circulating tumor DNA (ctDNA). In some embodiments, the ctDNA is present in a biological sample isolated from the subject. In some embodiments, the biological sample is a blood sample or a plasma sample. In some embodiments, the ctDNA is isolated from the biological sample prior to mutation identification.

In some embodiments of the diagnostic or treatment method described above, the tumor DNA is present in a tumor sample isolated from the subject. In some embodiments, the tumor DNA is isolated from the tumor sample prior to mutation identification.

In some embodiments of the diagnostic or treatment method described above, the one or more mutations are determined by sequencing. In some embodiments, the one or more mutations are determined using next-generation sequencing (NGS).

In some embodiments of the diagnostic or treatment method described above, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12. In some embodiments, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16. In some embodiments, the bispecific anti-EGFR/c-Met antibody is an IgG1 isotype.

In some embodiments of the diagnostic or treatment method described above, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments of the diagnostic or treatment method described above, the bispecific anti-EGFR/c-Met antibody comprises a biantennary glycan structure with a fucose content of about between 1% to about 15%.

In some embodiments of the diagnostic or treatment method described above, the bispecific anti-EGFR/c-Met antibody is administered intravenously to the subject. In some embodiments, the bispecific anti-EGFR/c-Met antibody is administered at a dose of between about 140 mg to about 2240 mg. In some embodiments, the bispecific anti-EGFR/c-Met antibody is administered at a dose of about 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1575 mg, 1600 mg, 2100 mg, or 2240 mg. In one embodiment, the bispecific anti-EGFR/c-Met antibody is administered at a dose of 1050 mg if the subject has a body weight of less than 80 kg. In one embodiment, the bispecific anti-EGFR/c-Met antibody is administered at a dose of 1400 mg if the subject has a body weight of greater than or equal to 80 kg.

In some embodiments of the diagnostic or treatment method described above, the bispecific anti-EGFR/c-Met antibody is administered subcutaneously or intradermally to the subject. In some embodiments, the bispecific anti-EGFR/c-Met antibody is administered subcutaneously or intradermally at a dose sufficient to achieve a therapeutic effect in the subject.

In some embodiments of the diagnostic or treatment method described above, the bispecific anti-EGFR/c-Met antibody is administered twice a week, once a week, once in two weeks, once in three weeks or once in four weeks. In one embodiment, the bispecific anti-EGFR/c-Met antibody is administered once a week. In another embodiment, the bispecific anti-EGFR/c-Met antibody is administered once in two weeks.

In some embodiments of the diagnostic or treatment method described above, the EGFR TKI administered in combination with the bispecific anti-EGFR/c-Met antibody is lazertinib. In some embodiments, the EGFR TKI administered in combination with the bispecific anti-EGFR/c-Met antibody is administered at a dose of between about 20 to about 320 mg. In some embodiments, the EGFR TKI administered in combination with the bispecific anti-EGFR/c-Met antibody is administered at a dose of about 240 mg. In some embodiments, the EGFR TKI administered in combination with the bispecific anti-EGFR/c-Met antibody is administered daily, every other day, twice a week, or once a week. In some embodiments, the EGFR TKI administered in combination with the bispecific anti-EGFR/c-Met antibody is administered daily. In some embodiments, the EGFR TKI administered in combination with the bispecific anti-EGFR/c-Met antibody is administered orally.

In some embodiments of the treatment method described above, the cancer therapy which does not include the combination therapy used in (i) is a platinum-based chemotherapy. In some embodiments, the platinum-based chemotherapy comprises carboplatin and/or cisplatin.

In some embodiments of the diagnostic or treatment method described above, the method further comprises obtaining a biological sample from the subject prior to step (a), wherein said biological sample comprises tumor DNA (e.g., ctDNA), and optionally purifying said tumor DNA (e.g., ctDNA) from said biological sample.

In another aspect, provided herein is a method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising a) determining expression level of EGFR or MET in a tumor sample obtained from the subject using immunohistochemistry (IHC),
b) determining a staining intensity score, on a scale from 0 to 3+, based on the expression level of EGFR or MET determined in step (a), and
c) (i) identifying the cancer in the subject as susceptible to the treatment with the combination therapy, when the staining intensity score is 3+, or (ii) identifying the cancer in the subject as not susceptible to the treatment with the combination therapy, when the staining intensity score is less than 3+.

In some embodiment of the above method, step (c) comprises (i) identifying the cancer in the subject as susceptible to the treatment with the combination therapy, when the staining intensity score is 3+ in greater than or equal to 25% of the cells of the tumor sample, or (ii) identifying the cancer in the subject as not susceptible to the treatment with the combination therapy, when the staining intensity score is 3+ in less than 25% cells of the tumor sample.

In another aspect, provided herein is a method for treating a cancer in a subject in need thereof, said method comprising
a) determining the expression level of EGFR or MET in a tumor sample obtained from the subject using immunohistochemistry (IHC),
b) determining a staining intensity score, on a scale from 0 to 3+, based on the expression level of EGFR or MET determined in step (a), and
c) (i) when the staining intensity score is 3+, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI); or (ii) when the staining intensity score is less than 3+, not administering to the subject the combination therapy used in (i) or administering to the subject a cancer therapy which does not include the combination therapy used in (i).

In some embodiment of the above method, step (c) comprises (i) when the staining intensity score is 3+ in greater than or equal to 25% of the cells of the tumor sample, administering to the subject a therapeutically effective amount of the combination therapy; or (ii) when the staining intensity score is 3+ in less than 25% cells of the tumor sample, not administering to the subject the combination therapy used in (i) or administering to the subject a cancer therapy which does not include the combination therapy used in (i).

In another aspect, provided herein is a method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising a) determining the expression level of EGFR and MET in a tumor sample obtained from the subject using immunohistochemistry (IHC),
b) calculating a combined H score based on the expression level of EGFR and MET determined in step (a), and
c) (i) identifying the cancer in the subject as susceptible to the treatment with the combination therapy, when the combined H score is greater than or equal to 400, or (ii) identifying the cancer in the subject as not susceptible to the treatment with the combination therapy, when the combined H score is less than 400.

In another aspect, provided herein is a method for treating a cancer in a subject in need thereof, said method comprising
a) determining the expression level of EGFR and MET in a tumor sample obtained from the subject using immunohistochemistry (IHC),
b) calculating a combined H score based on the expression level of EGFR and MET determined in step (a), and
c) (i) when the combined H score is greater than or equal to 400, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI); (ii) when the combined H score is less than 400, not administering to the subject the combination therapy used in (i) or administering to the subject a cancer therapy which does not include the combination therapy used in (i).

In some embodiments of the diagnostic or treatment method described above, the cancer is a lung cancer, such as a non-small cell lung cancer (NSCLC).

In some embodiments of the diagnostic or treatment method described above, the cancer in the subject is resistant to treatment with an EGFR TKI which is not the same as the EGFR TKI used in the combination therapy. In some embodiments, the EGFR TKI to which the cancer is resistant is selected from osimertinib, erlotinib, afatinib, rociletinib, olmutinib, and any combination thereof. In one embodiment, the EGFR TKI to which the cancer is resistant is osimertinib.

In some embodiments of the diagnostic or treatment method described above, the subject is chemotherapy naïve.

In some embodiments of the diagnostic or treatment method described above, the tumor of the subject has at least one EGFR-activating mutation. In some embodiments, the EGFR-activating mutation is selected from exon 19 deletions, L858R, and T790M.

In some embodiments of the diagnostic or treatment method described above, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12. In some embodiments, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16. In some embodiments, the bispecific anti-EGFR/c-Met antibody is an IgG1 isotype.

In some embodiments of the diagnostic or treatment method described above, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments of the diagnostic or treatment method described above, the bispecific anti-EGFR/c-Met antibody comprises a biantennary glycan structure with a fucose content of about between 1% to about 15%.

In some embodiments of the diagnostic or treatment method described above, the bispecific anti-EGFR/c-Met antibody is administered intravenously to the subject. In some embodiments, the bispecific anti-EGFR/c-Met antibody is administered at a dose of between about 140 mg to about 2240 mg. In some embodiments, the bispecific anti-EGFR/c-Met antibody is administered at a dose of about 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1575 mg, 1600 mg, 2100 mg, or 2240 mg. In one embodiment, the bispecific anti-EGFR/c-Met antibody is administered at a dose of 1050 mg if the subject has a body weight of less than 80 kg. In one embodiment, the bispecific anti-EGFR/c-Met antibody is administered at a dose of 1400 mg if the subject has a body weight of greater than or equal to 80 kg.

In some embodiments of the diagnostic or treatment method described above, the bispecific anti-EGFR/c-Met antibody is administered subcutaneously or intradermally to the subject. In some embodiments, the bispecific anti-EGFR/c-Met antibody is administered subcutaneously or intradermally at a dose sufficient to achieve a therapeutic effect in the subject.

In some embodiments of the diagnostic or treatment method described above, the bispecific anti-EGFR/c-Met antibody is administered twice a week, once a week, once in two weeks, once in three weeks or once in four weeks. In one embodiment, the bispecific anti-EGFR/c-Met antibody is administered once a week. In another embodiment, the bispecific anti-EGFR/c-Met antibody is administered once in two weeks.

In some embodiments of the diagnostic or treatment method described above, the EGFR TKI administered in combination with the bispecific anti-EGFR/c-Met antibody is lazertinib. In some embodiments, the EGFR TKI administered in combination with the bispecific anti-EGFR/c-Met antibody is administered at a dose of between about 20 to about 320 mg. In some embodiments, the EGFR TKI administered in combination with the bispecific anti-EGFR/c-Met antibody is administered at a dose of about 240 mg. In some embodiments, the EGFR TKI administered in combination with the bispecific anti-EGFR/c-Met antibody is administered daily, every other day, twice a week, or once a week. In some embodiments, the EGFR TKI administered in combination with the bispecific anti-EGFR/c-Met antibody is administered daily. In some embodiments, the EGFR TKI administered in combination with the bispecific anti-EGFR/c-Met antibody is administered orally.

In some embodiments of the treatment method described above, the cancer therapy which does not include the combination therapy used in (i) is a platinum-based chemotherapy. In some embodiments, the platinum-based chemotherapy comprises carboplatin and/or cisplatin.

In some embodiments of the diagnostic or treatment method described above, the method further comprises a tumor sample from the subject prior to step (a).

In another aspect, provided herein is a diagnostic kit comprising (i) one or more reagents for determining the presence of one or more mutations in tumor DNA from a subject with a cancer, and (ii) optionally packaging and/or instructions for use, wherein the one or more mutations are selected from mutations in one or more genes from the RAS/RAF/MEK pathway and mutations in PIK3CA.

In some embodiments of the kit described above, the one or more genes from RAS/RAF/MEK pathway are FGFR3, KRAS, BRAF, ERBB2, ALK, NRAS, PDGFRA and/or RET. In some embodiments, the mutations in one or more genes from RAS/RAF/MEK pathway comprise FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions. In some embodiments, the KRAS G12X mutations are KRAS G12D, KRAS G12A, KRAS G12C, and KRAS G12V.

In some embodiments of the kit described above, the mutations in PIK3CA comprise PIK3CA E545K.

In some embodiments of the kit described above, the one or more mutations are further selected from mutations in one or more genes from the WNT/b-catenin pathway. In some embodiments, the one or more genes from WNT/b-catenin pathway are APC and CTNNB1. In some embodiments, the mutations in one or more genes from WNT/b-catenin pathway comprise APC Q1469, APC R405, APC S713, CTNNB1 S33P, CTNNB1 S37C, CTNNB1 S37F, and CTNNB1 S45P.

In another aspect, provided herein is a diagnostic kit comprising (i) one or more reagents for determining the presence of one or more mutations in tumor DNA from a subject with a cancer, and (ii) optionally packaging and/or instructions for use, wherein the one or more mutations are selected from the following two groups:
(1) PIK3CA E545K, PIK3CA E542K/V, PIK3CA H1047R, PIK3CA amplification, KRAS G12V/C/D/X (X being any amino acid other than G, V, C and D), KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletions, PTEN N48K, CDKN2A G101W, CDKN2B mutations, ALK fusions, FGFR3-TACC3 fusion, TPM3-NTRK1 fusion, RET fusions, BRAF fusions, and other oncogenic fusion events; and
(2) EGFR C797S, EGFR L792H, EGFR amplification, EGFR G796S, EGFR L718X (X being any amino acid), EGFR E709K, EGFR G724S, MET amplification, and MET exon 14 skipping (METex14) mutations.

In some embodiments of the kit described above, the HER2 oncogenic alterations comprise HER2 Y772_A775 duplication, HER2 L755M/S/W and HER2 S310F/Y. In some embodiments, the PTEN deletions comprise PTEN I33del and PTEN I14del. In some embodiments, the ALK fusions comprise SQSTM1-ALK fusion and EML4-ALK fusion. In some embodiments, the RET fusions comprise CCDC6-RET fusion, KIF5B-RET fusion, and NCOA4-RET fusion.

In some embodiments of the kit described above, the tumor DNA is circulating tumor DNA (ctDNA). In some embodiments, the ctDNA is present in a biological sample isolated from the subject. In some embodiments, the biological sample is a blood sample or a plasma sample. In some embodiments, the tumor DNA is present in a tumor sample isolated from the subject.

In some embodiments of the kit described above, the kit further comprises one or more reagents for purifying said tumor DNA from said biological sample from the subject.

In some embodiments of the kit described above, the one or more reagents can be used with a sequencing technique, such as next-generation sequencing (NGS), to determine the one or more mutations.

In another aspect, provided herein is a diagnostic kit comprising (i) one or more reagents for determining the expression level of EGFR and/or MET in a tumor sample from a subject with a cancer, and (ii) optionally packaging and/or instructions for use. In some embodiments, the one or more reagents can be used with immunohistochemistry (IHC) to determine the expression level of EGFR and/or MET.

These and other aspects described herein will be apparent to those of ordinary skill in the art in the following description, claims and drawings.

### BRIEF DESCRIPTION OF THE DRAWINGS

FIG. 1 shows schematic representations of the structure of amivantamab and lazertinib (left), and detailed description of the mechanism of action (MOA) for amivantamab (right).

FIG. 2 shows a schematic representation of the progression of acquired resistance to osimertinib in epidermal growth factor receptor mutant (EGFRm) non-small cell lung cancer (NSCLC). Sequencing of a single tumor lesion may not reveal heterogenous pattern or co-occurring mutations at resistance. In this sense, next-generation sequencing (NGS) of circulating tumor DNA (ctDNA) from plasma samples may be more useful (Papadimitrakopoulou et al., Annals of Oncol 29:V111741, 2018; Ramalingam et al., Annals of Oncol 29:VII1740, 2018). ctDNA, circulating tumor DNA; Exon19del, exon 19 deletion.

FIG. 3 shows the study design for CHRYSALIS phase 1 trial corresponding to the combination cohort (Cho et al., Ann Oncol 31:S813, 2020). ^{a}One or more alterations were detected in 42/44 ctDNA and 29/45 tumor NGS analyses. C, cycle; IHC, immunohistochemistry; QW, weekly; Q2W, every 2 weeks; RP2CD, recommended phase 2 combination dose.

FIG. 4 shows a summary chart of patient demographics and baseline disease characteristics. ^{a}Based on local testing, central testing identified Exon 19 deletion.

FIGS. 5A-5B show durable responses observed with combined amivantamab plus lazertinib (combined amivantamab/lazertinib treatment) with manageable safety. FIG. 5A displays a plot of the percentage change from baseline in sum of target lesion diameters (SoD) across months of the study. Four patients did not have post-baseline disease assessments and are not included in the plot. FIG. 5B shows a summary chart of the investigator-assessed response (N=45 patients). The safety profile as demonstrated by these data are consistent with previous experience with amivantamab plus lazertinib by the inventors (Cho et al., Ann Oncol 31:S813, 2020). The most common adverse events (AE) were infusion-related reaction (IRR; 78%), rash (acneiform dermatitis, 51%+rash, 27%), and paronychia (49%), the majority of which were grade 1-2. Of treatment-related instances, 16% were grade ≥3 AE, 4% were discontinuations, and 18% were dose reductions. CBR, clinical benefit rate (CR, PR, or SD≥11 weeks); CR, complete response; IRR, infusion-related reaction; mDOR, median duration of response; mDOT, median duration of treatment; mF/U, median follow-up; mPFS, median progression-free survival; NE, not evaluable; NR, not reached; ORR, overall response rate; PD, progressive disease; PR, partial response; SD, stable disease; UNK, unknown.

FIGS. 6A-6B demonstrate response among patients with identified EGFR/MET-based resistance. FIG. 6A displays a plot of the best percentage change in tumor volume for EGFR-based resistance, MET-based-resistance, and EGFR plus MET (EGFR+MET)-based resistance groups. Additional alterations to RAS/RAF pathway—(†), mTOR pathway—(A), cell cycle—(¥) and fusion event—(¤) related genes are also indicated, as well as instances of no tumor NGS (*). FIG. 6B shows a summary chart of the genetic alterations determined for the EGFR-based, MET-based, and additional resistance groups. Seventeen out of the total 45 patients were identified with either EGFR/MET-based resistance by NGS^{a} (ctDNA/tissue). Overall response rate (ORR) in this subgroup was 47% (8/17 patients); median duration of response (mDOR) was 10.4 months; clinical benefit rate (CBR) was 82%; and, median progression-free survival (mPFS) was 6.7 months. ^{a}Genomic analysis used Guardant360 for ctDNA NGS and ThermoFisher for tissue NGS; ^{b}EGFR amplification (copy number variation, CNV≥7) and MET amplification (CNV≥3) were based on tumor NGS; other amplifications were based on tumor NGS (CNV≥7) or ctDNA NGS (CNV≥3). Single nucleotide variants, insertion/deletions, and insertion call threshold was ≥5% allele frequency with >250 reads. ^{c}Eight patients had ≥1 alteration. Amp, amplification; CNV, copy number variation.

FIGS. 7A-7B demonstrate response among patients without identified EGFR/MET-based resistance. FIG. 7A displays a plot of the best percentage change in tumor volume for unknown resistance mechanism and EGFR/MET-independent resistance groups. Additional alterations to RAS/RAF pathway—(†), mTOR pathway—(Δ), cell cycle—(¥) and fusion event—(¤) related genes are also indicated, as well as instances of no tumor NGS (*). FIG. 7B shows a summary chart of exemplary genetic alterations determined for the EGFR/MET-independent group. According to the present patient stratification method, in the absence of such mutations, a patient may be a candidate for amivantamab and lazertinib combination treatments disclosed herein. Further, based on methods disclosed herein, patients with a non-EGFR non-MET (i.e., EGFR/MET-independent) mechanism of resistance, as exemplified in the present Figure, would be excluded from treatment with amivantamab in combination with lazertinib treatments. These data indicate that such patients are at a low probability of responding to this combination, and given the current standard of care, can be treated with e.g., platinum-based chemotherapy. Specifically, among the 28 patients who did not have an identified EGFR/MET-based resistance by NGS^{a}, the overall response rate (ORR) was 29% (8/28 patients), median duration of response (mDOR) was 8.3 months, clinical benefit rate (CBR) was 54%; and median progression-free survival (mPFS) was 4.1 months. All 8 responders in those without identified EGFR/MET-based resistance were unknown resistance by NGS. ^{a}Genomic analysis used Guardant360 for ctDNA NGS and ThermoFisher for tissue NGS. ^{b}Two patients had ≥1 alteration. NE, not evaluable (no post-baseline assessment for 4 patients).

FIG. 8 shows the response among patients with EGFR/MET expression as identified by immunohistochemical (IHC) staining approaches. IHC identified patients regardless of the genetic resistance mechanisms. Of the 45 patients, 20 patients had tumor biopsy that was sufficient for IHC staining after next-generation sequencing (NGS). Ten patient biopsy samples were immune-positive (IHC+) for EGFR/MET, exhibiting a combined EGFR plus MET (EGFR+MET) H score ≥400. The remaining ten patient biopsy samples were defined as IHC−. IHC+ patients had an overall response rate (ORR) of 90% (9/10 patients); median duration of response (mDOR) of 9.7 months; clinical benefit rate (CBR) of 100%; and median progression-free survival (mPFS) of 12.5 months. The plot (top) displays the best percentage change in tumor volume for the IHC+ and IHC− groups, and the chart (bottom) shows the corresponding patient stratification for EGFR-based resistance, MET-based resistance, EGFR/MET-independent resistance, and unknown resistance mechanism groups. Five responders in the IHC+ group had an unknown genetic mechanism. A high representation of positive responders (PRs) was observed in the IHC+ group, and was associated with greater percentage reduction in tumor volume. These data suggest that IHC of MET and/or EGFR, indicative of high expression, is a positive predictor of treatment response to combined amivantamab/lazertinib treatment. NE, not evaluable (no postbaseline assessment for 2 patients).

FIG. 9 shows an exemplary schematic diagram of the CHRYSALIS-2 study design. This phase 1/1b expansion cohort study seeks to validate biomarkers disclosed herein in a new cohort requiring tumor biopsy at entry among post-osimertinib EGFRm NSCLC (NCT04077463; Poster TPS9132, “CHRYSALIS-2: A phase 1/1b study of lazertinib as monotherapy and in combination with amivantamab in patients with EGFR mutant NSCLC”).

FIG. 10A-10B show Kaplan-Meier progression-free survival curves (FIG. 10A) and waterfall plots for target lesion tumor size (FIG. 10B) in patient population with and without pathogenic alterations in RAS-RAF-MEK pathways or pathogenic PIK3CA-E545K (“NGS1”).

FIG. 11A-11B show Kaplan-Meier progression-free survival curves (FIG. 11A) and waterfall plots for target lesion tumor size (FIG. 11B) in patient population with and without pathogenic alterations in RAS-RAF-MEK pathways or pathogenic PIK3CA-E545K, or pathogenic alterations in WNT/beta-catenin pathways (“NGS2”).

FIG. 12A-12B show Kaplan-Meier progression-free survival curves (FIG. 12A) and waterfall plots for target lesion tumor size (FIG. 12B) in patient population with and without pathogenic alterations in RAS-RAF-MEK pathways or pathogenic PIK3CA-E545K, or pathogenic alterations in WNT/beta-catenin pathways or wildtype EGFR-drivers (“NGS3”). “Exclude”=NGS3 positive group; “Include”=NGS3 negative group.

FIG. 13A-13B show swimlane plots of best change from baseline in sum of diameters (SoD) of target lesion for populations dichotomized by IHC1 (EGFR) classification (FIG. 13A—IHC1 Positive; FIG. 13B—IHC1 Negative).

FIGS. 14A-14B show swimlane plots of best change from baseline in SoD of target lesion for populations dichotomized by IHC2 (MET) classification (FIG. 14A—IHC2 Positive; FIG. 14B—IHC2 Negative).

FIG. 15A-15B show progression-free survival curves by IHC1 (EGFR) classification (FIG. 15A) or IHC2 (MET) classification (FIG. 15B) in Response Evaluable at RP2CD Analysis Set in Combination Therapy.

### DETAILED DESCRIPTION

### Definitions

All publications, including but not limited to patents and patent applications, cited in this specification are herein incorporated by reference as though fully set forth.

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“Co-administration,” “administration with,” “administration in combination with,” “in combination with” or the like, encompass administration of the selected therapeutics or drugs to a single patient, and are intended to include treatment regimens in which the therapeutics or drugs are administered by the same or different route of administration or at the same or different time.

“Isolated” refers to a homogenous population of molecules (such as synthetic polynucleotides, polypeptides vectors or viruses) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent”, “preventing”, “prevention”, or “prophylaxis” of a disease or disorder means preventing that a disorder occurs in subject.

“Diagnosing” or “diagnosis” refers to methods to determine if a subject is suffering from a given disease or condition or may develop a given disease or condition in the future or is likely to respond to treatment for a prior diagnosed disease or condition, i.e., stratifying a patient population on likelihood to respond to treatment. Diagnosis is typically performed by a physician based on the general guidelines for the disease to be diagnosed or other criteria that indicate a subject is likely to respond to a particular treatment.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Newly diagnosed” refers to a subject who has been diagnosed with cancer (e.g., EGFR or c-Met expressing cancer) but has not yet received treatment (e.g., treatment for lung cancer).

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Cancer” refers to an abnormal growth of cells which tend to proliferate in an uncontrolled way and, in some cases, to metastasize (spread) to other areas of a patient's body.

“EGFR or c-Met expressing cancer” refers to cancer that has detectable expression of EGFR or c-Met or has EGFR or c-Met mutation or amplification. EGFR or c-Met expression, amplification and mutation status can be detected using know methods, such as sequencing, fluorescent in situ hybridization, immunohistochemistry, flow cytometry or western blotting.

“Epidermal growth factor receptor” or “EGFR” refers to the human EGFR (also known as HER1 or ErbB1 (Ullrich et al., Nature 309:418-425, 1984)) having the amino acid sequence shown in GenBank accession number NP_005219, as well as naturally-occurring variants thereof.

“Hepatocyte growth factor receptor” or “c-Met” as used herein refers to the human c-Met having the amino acid sequence shown in GenBank Accession No: NP_001120972 and natural variants thereof.

“Bispecific anti-EGFR/c-Met antibody” or “bispecific EGFR/c-Met antibody” refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

“Specific binding” or “specifically binds” or “specifically binding” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 5×10⁻⁸M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The dissociation constant may be measured using known protocols. Antibodies that bind to the antigen or the epitope within the antigen may, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody binds one antigen or one epitope, a bispecific antibody binds two distinct antigens or two distinct epitopes.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Complementarity determining regions” (CDR) are antibody regions that bind an antigen. CDRs may be defined using various delineations such as Kabat (Wu et al. (1970) J Exp Med 132: 211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton (1996) J Bmol Biol 263: 800-15). The correspondence between the various delineations and variable region numbering are described (see e.g., Lefranc et al. (2003) Dev Comp Immunol 27: 55-77; Honegger and Pluckthun, (2001) J Mol Biol 309:657-70; International ImMunoGeneTics (IMGT) database; Web resources, http://imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Antigen binding fragment” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Recombinant” refers to DNA, antibodies and other proteins that are prepared, expressed, created or isolated by recombinant means when segments from different sources are joined to produce recombinant DNA, antibodies or proteins.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Antagonist” or “inhibitor” refers to a molecule that, when bound to a cellular protein, suppresses at least one reaction or activity that is induced by a natural ligand of the protein. A molecule is an antagonist when the at least one reaction or activity is suppressed by at least about 20%, 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist.

“PD-(L)1 axis inhibitor” refers to a molecule that inhibits PD-1 downstream signaling. PD-(L)1 axis inhibitor may be a molecule that binds PD-1, PD-L1 or PD-L2.

“Biological sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids and the like, tissue biopsies, tumor tissue biopsies, tumor tissue samples, fine needle aspirations, surgically resected tissue, organ cultures or cell cultures. As a non-limiting example, the biological sample is a blood sample. As another non-limiting example, the biological sample is a plasma sample. As yet another non-limiting example, the biological sample is a tumor sample. In some embodiments, the biological sample is circulating tumor DNA (ctDNA) that may be isolated from various other biological samples disclosed herein such as, but not limited to, a blood or plasma sample. In some embodiments, the biological sample is tumor DNA that may be isolated from, e.g., a tumor sample.

“Low fucose” or “low fucose content” as used in the application refers to antibodies with fucose content of about between 1%-15%.

“Normal fucose” or “normal fucose content” as used herein refers to antibodies with fucose content of about over 50%, typically about over 80% or over 85%.

### Methods of the Disclosure

### Diagnostic Methods

In one aspect, the present disclosure provides a method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI).

In some embodiments, the method comprises a) determining the presence of one or more mutations in tumor DNA obtained from the subject, wherein the one or more mutations are selected from mutations in one or more genes from the RAS/RAF/MEK pathway and mutations in Phosphatidylinositol-4,5-Bisphosphate 3-Kinase Catalytic Subunit Alpha (PIK3CA); and b) (i) identifying the cancer in the subject as susceptible to the treatment with the combination therapy, when tumor DNA from said subject has no said mutations, or (ii) identifying the cancer in the subject as not susceptible to the treatment with the combination therapy, when tumor DNA from said subject has one or more said mutations.

In some embodiments, the one or more mutations are further selected from mutations in one or more genes from the WNT/b-catenin pathway.

The mutations associated with RAS/RAF/MEK pathway or WNT/b-catenin pathway, or mutations in PIK3CA may include those pathogenic mutations known in the art.

In some embodiments, the mutations may be found in one or more genes from RAS/RAF/MEK pathway, such as but not limited to, fibroblast growth factor receptor 3 (FGFR3), Kirsten rat sarcoma virus oncogene (KRAS), v-raf murine sarcoma viral oncogene homolog B1 (BRAF), Erb-B2 receptor tyrosine kinase 2 (ERBB2), anaplastic lymphoma receptor tyrosine kinase (ALK), neuroblastoma-RAS (NRAS), platelet-derived growth factor receptor A (PDGFRA) and/or Ret proto-oncogene (RET).

In some embodiments, the mutations in one or more genes from RAS/RAF/MEK pathway may include, but are not limited to, FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions.

In some embodiments, the KRAS G12X mutations are KRAS G12D, KRAS G12A, KRAS G12C, and KRAS G12V.

In some embodiments, mutations in BRAF may include those described in R. Yaeger et al., Targeting Alterations in the RAF-MEK Pathway. Cancer Discov (2019) 9 (3): 329-341, which is incorporated herein by reference in its entirety, for example, but not limited to, V600E/K/D/R/M, P367L/S, G464V/E, L485W, N486_A489delinsK, N486_P490del, E586K, L597Q/R/S/V, T599TT/TS, T5991/K, K601E/N/T, K601_S602delinsNT, BRAF kinase duplication, fusions of BRAF kinase domain, D287H, V459L, G466A/E/V, S467L, G469E, N581I/S/T, D594A/G/H/N, F595L, G596D/R.

In some embodiments, mutations in BRAF may include those described in H. Yang et al., New Horizons in KRAS-Mutant Lung Cancer: Dawn After Darkness. Front. Oncol., 25 Sep. 2019, which is incorporated herein by reference in its entirety, for example, but not limited to, E3K, G12CN/D/A/S/R/F, G13C/D/EN/R, V14I, Q61L/E/H/R, F61L, L19F, D33E, T58I, A59T, A146P/V/T, C118S, A59_G60delinsGV.

In some embodiments of the diagnostic or treatment method described above, wherein the mutations in PIK3CA comprise PIK3CA E545K.

In some embodiments, the mutations may be found in one or more genes from WNT/b-catenin pathway, such as but not limited to, APC and CTNNB1.

In some embodiments, the mutations in one or more genes from WNT/b-catenin pathway comprise APC Q1469, APC R405, APC 5713, CTNNB1 S33P, CTNNB1 S37C, CTNNB1 S37F, and CTNNB1 S45P.

In some embodiments, the present disclosure also provides a method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising a) determining the presence of one or more mutations in tumor DNA (e.g., circulating tumor DNA (ctDNA)) obtained from the subject, wherein the one or more mutations are selected from the following two groups: (1) Phosphatidylinositol-4,5-Bisphosphate 3-Kinase Catalytic Subunit Alpha (PIK3CA) E545K, PIK3CA E542K/V, PIK3CA H1047R, PIK3CA amplification, Kirsten rat sarcoma viral oncogene (KRAS) G12V/C/D/X (X being any amino acid other than G, V, C and D), KRAS amplification, v-raf murine sarcoma viral oncogene homolog B1 (BRAF) V600E, BRAF amplification, Cyclin D1 (CCND1) amplification, Cyclin D2 (CCND2) amplification, Cyclin E1 (CCNE1) amplification, Cyclin Dependent Kinase 4 (CDK4) amplification, Cyclin Dependent Kinase 6 (CDK6) amplification, Erb-B2 Receptor Tyrosine Kinase 2 (HER2) amplification, HER2 oncogenic alterations, Phosphatase And Tensin Homolog (PTEN) deletion, PTEN N48K, Cyclin Dependent Kinase Inhibitor 2A (CDKN2A) G101W, CDKN2B mutations, Anaplastic Lymphoma Receptor Tyrosine Kinase (ALK) fusions, Fibroblast Growth Factor Receptor 3-Transforming Acidic Coiled-Coil Containing Protein 3 (FGFR3-TACC3) fusion and other fusions (e.g., TPM3-NTRK1 fusion), Ret Proto-Oncogene (RET) fusions, v-raf murine sarcoma viral oncogene homolog B1 (BRAF) fusions, and other oncogenic fusion events; (2) EGFR C797S, EGFR L792H, EGFR amplification, EGFR G796S, EGFR L718X (X being any amino acid), EGFR E709K, EGFR G724S, MET amplification, and MET exon 14 skipping (METex14) mutations; and b) (i) identifying the cancer in the subject as susceptible to the treatment with the combination therapy, when tumor DNA (e.g., ctDNA) from said subject has either no mutations from group (1), or has one or more mutations from group (1) and one or more mutations from group (2), or (ii) identifying the cancer in the subject as not susceptible to the treatment with the combination therapy, when tumor DNA (e.g., ctDNA) from said subject has one or more mutations from group (1) and no mutations from group (2).

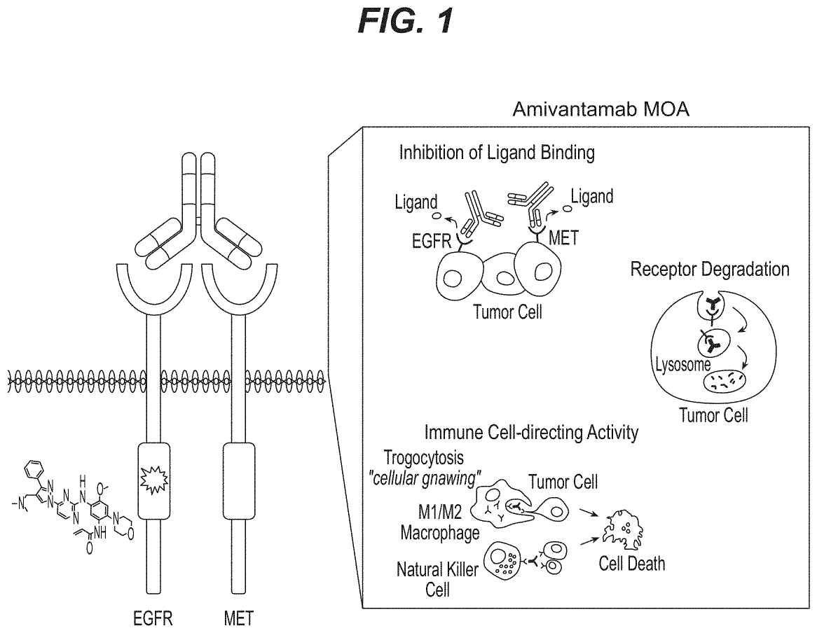

Non-limiting examples of HER2 oncogenic alterations include HER2 Y772_A775 duplication, HER2 L755M/S/W and HER2 S310F/Y. Non-limiting examples of PTEN deletions include PTEN I33del and PTEN4del. Non-limiting examples of ALK fusions include SQSTM1-ALK fusion and EML4-ALK fusion. Non-limiting examples of RET fusions include CCDC6-RET fusion, KIF5B-RET fusion, and NCOA4-RET fusion. Non-limiting examples of BRAF fusions include those described in Ross et al., Int. J. Cancer: 138, 881-890 (2016), which is incorporated herein by reference in its entirety, such as KIAA1549-BRAF, MKRN1-BRAF, TRIM24-BRAF, AGAP3-BRAF, ZC3HAV1-BRAF, AKAP9-BRAF, CCDC6-BRAF, AGK-BRAF, EPS15-BRAF, NUP214-BRAF, ARMC10-BRAF, BTF3L4-BRAF, GHR-BRAF, ZNF767-BRAF, CCDC91-BRAF, DYNC1I2-BRAF, ZKSCAN1-BRAF, GTF2I-BRAF, MZT1-BRAF, RAD18-BRAF, CUX1-BRAF, SLC12A7-BRAF, MYRIP-BRAF, SND1-BRAF, NUB1-BRAF, KLHL7-BRAF, TANK-BRAF, RBMS3-BRAF, STRN3-BRAF, STK35-BRAF, ETFA-BRAF, SVOPL-BRAF, and JHDM1D-BRAF. Other oncogenic fusion events include, but are not limited to, those disclosed in FIG. 1 of Gao et al., Cell Rep. 2018 Apr. 3; 23(1): 227-238.e3., which is incorporated herein by reference in its entirety.

In addition to the specifically described mutations, the mutations can also be selected as follows:
Annotate genes as oncogenes or tumor suppressor genes based on the COSMIC cancer gene census (Sondk et al., Nature Reviews Cancer volume 18, 696-705 (2018), which is incorporated herein by reference in its entirety).
For oncogenes, activating short variants are identified if they are:
   listed as oncogenic or likely oncogenic in OncoKb (Chakravarty et al., JCO Precision Oncology. 2017:1, 1-16, which is incorporated herein by reference in its entirety)
   found at a cancer hotspot, i.e., mutated statistically significantly more often than expected by chance, as listed in cancer hotspots (Chang et al., Cancer Discov. 2018 February; 8(2):174-183, which is incorporated herein by reference in its entirety)
   an explicitly known activating mutation (i.e., KRAS G12C)
A limited set of oncogenes are evaluated for copy number and fusions on the Guardant 360 panel. These are also classified as activating if the copy number is greater than 3 or if any fusion is detected.
For tumor suppressors, inactivating short variants are identified if they are:
   Listed as oncogenic or likely oncogenic in OncoKb
   Found at a cancer hotspot in cancer hotspots
   Result in a truncating mutation, i.e., nonsense, frameshift or splice site mutation
   An explicitly known inactivating mutation

In another aspect, the present disclosure provides a method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising a) determining the expression level of EGFR and MET in a tumor sample obtained from the subject using immunohistochemistry (IHC), b) calculating a combined H score based on the expression level of EGFR and MET determined in step (a), and c) (i) identifying the cancer in the subject as susceptible to the treatment with the combination therapy, when the combined H score is greater than or equal to 400, or (ii) identifying the cancer in the subject as not susceptible to the treatment with the combination therapy, when the combined H score is less than 400.

A staining intensity value may be assigned to a tumor sample as a semiquantitative approach useful for analyses of immunohistochemical results. Such approach is well know in the art. The staining intensity is assigned on a scale from 0 to 3+(0, 1+, 2+, or 3+), wherein 0 is assigned when staining is not visible or detectable, and 3+ is assigned to the highest intensity staining, and may be determined for each cell in a fixed field. The tumor sample can be fixed in formalin paraffin embedded tissue (FFPE).

An H score (or histo score) may be assigned to a tumor sample as a semiquantitative approach useful for analyses of immunohistochemical results (Hirsch F R et al., J Clin Oncol 21:3798-3807, 2003; John T et al., Oncogene 28:S14-S23, 2009). As a non-limiting example, membrane staining intensity (0, 1+, 2+, or 3+) may be determined for each cell in a fixed field. The tumor sample can be fixed in formalin paraffin embedded tissue (FFPE). In some embodiments, the H score may be based on a predominant staining intensity. In some embodiments, the H score may include the sum of individual H scores for each intensity level seen. As a non-limiting example, the percentage of cells at each staining intensity level may be calculated, and finally, an H score may be assigned using the following exemplary formula: [1×(% cells 1+)+2×(% cells 2+)+3×(% cells 3+)]. The final calculated H score, ranging from 0 to 300, may give more relative weight to higher-intensity membrane staining in a given tumor sample. In some embodiments, the tumor sample may be considered either positive or a negative on the basis of a specific discriminatory threshold.

A “combined H score”, as referred to herein, can be generated by adding an H score calculated from the analysis of one biomarker (e.g., EGFR expression) to an H score calculated from the analysis of a second biomarker (e.g., MET expression). Accordingly, the combined H score can have a range of 0 to 600.

In another aspect, the present disclosure provides a method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising a) determining expression level of EGFR or MET in a tumor sample obtained from the subject using immunohistochemistry (IHC), b) determining a staining intensity score, on a scale from 0 to 3+, based on the expression level of EGFR or MET determined in step (a), and c) (i) identifying the cancer in the subject as susceptible to the treatment with the combination therapy, when the staining intensity score is 3+, or (ii) identifying the cancer in the subject as not susceptible to the treatment with the combination therapy, when the staining intensity score is less than 3+.

In some embodiments, the method comprises in step c) identifying the cancer in the subject as susceptible to the treatment with the combination therapy, when the intensity score is 3+ in greater than or equal to 5%, 7.5%, 10%, 12.5%, 15%, 17.5%, 20%, 22.5%, 25%, 27.5%, 30%, 32.5%, 35%, 37.5%, 40%, 42.5%, 45%, 47.5% or 50% of the cells of the tumor sample. Accordingly, step c) may comprise (ii) identifying the cancer in the subject as not susceptible to the treatment with the combination therapy, when the intensity score is 3+ in less than 5%, 7.5%, 10%, 12.5%, 15%, 17.5%, 20%, 22.5%, 25%, 27.5%, 30%, 32.5%, 35%, 37.5%, 40%, 42.5%, 45%, 47.5% or 50% cells of the tumor sample.

In one embodiment, the method comprises in step c) identifying the cancer in the subject as susceptible to the treatment with the combination therapy, when the intensity score is 3+ in greater than or equal to 25% of the cells of the tumor sample; or (ii) identifying the cancer in the subject as not susceptible to the treatment with the combination therapy, when the intensity score is 3+ in less than 25% cells of the tumor sample.

In various embodiments, the cancer assessed by the methods of the present disclosure is a lung cancer. In some embodiments, the lung cancer is a non-small cell lung cancer (NSCLC). In some embodiments, the cancer in the subject is resistant to treatment with an EGFR TKI which is not the same as the EGFR TKI used in the combination therapy. Non-limiting examples of the EGFR TKI to which the cancer may be resistant are osimertinib, erlotinib, afatinib, rociletinib, olmutinib, and any combination thereof. In some embodiments, the EGFR TKI to which the cancer may be resistant is osimertinib.

In some embodiments, the subject is resistant or has acquired resistance to an EGFR inhibitor. Exemplary EGFR inhibitors for which cancer may acquire resistance are anti-EGFR antibodies cetuximab (ERBITUX®), pantinumumab (VECTIBIX®), matuzumab, nimotuzumab, small molecule EGFR inhibitors erlotinib (TARCEVA®), gefitinib (IRESSA®), EKB-569 (pelitinib, irreversible EGFR TKI), pan-ErbB and other receptor tyrosine kinase inhibitors, lapatinib (EGFR and HER2 inhibitor), pelitinib (EGFR and HER2 inhibitor), vandetanib (ZD6474, ZACTIMA™, EGFR, VEGFR2 and RET TKI), PF00299804 (dacomitinib, irreversible pan-ErbB TKI), CI-1033 (irreversible pan-erbB TKI), afatinib (BIBW2992, irreversible pan-ErbB TKI), AV-412 (dual EGFR and ErbB2 inhibitor), EXEL-7647 (EGFR, ErbB2, GEVGR and EphB4 inhibitor), CO-1686 (irreversible mutant-selective EGFR TKI), AZD9291 (irreversible mutant-selective EGFR TKI), mobocertinib (TAK788, irreversible EGFR TKI), poziotinib (irreversible pan-EGFR or HER TKI) and HM-272 (neratinib, irreversible EGFR/ErbB2 inhibitor).

Various qualitative and/or quantitative methods may be used to determine if a subject is resistant, has developed or is susceptible to developing a resistance to treatment with an anti-cancer therapy. Symptoms that may be associated with resistance to an anti-cancer therapy include a decline or plateau of the well-being of the patient, an increase in the size of a tumor, arrested or slowed decline in growth of a tumor, and/or the spread of cancerous cells in the body from one location to other organs, tissues or cells. Re-establishment or worsening of various symptoms associated with cancer may also be an indication that a subject has developed or is susceptible to developing resistance to an anti-cancer therapy, such as anorexia, cognitive dysfunction, depression, dyspnea, fatigue, hormonal disturbances, neutropenia, pain, peripheral neuropathy, and sexual dysfunction. The symptoms associated with cancer may vary according to the type of cancer. For example, symptoms associated with cervical cancer may include abnormal bleeding, unusual heavy vaginal discharge, pelvic pain that is not related to the normal menstrual cycle, bladder pain or pain during urination, and bleeding between regular menstrual periods, after sexual intercourse, douching, or pelvic exam. Symptoms associated with lung cancer may include persistent cough, coughing up blood, shortness of breath, wheezing chest pain, loss of appetite, losing weight without trying and fatigue. Symptoms for liver cancer may include loss of appetite and weight, abdominal pain, especially in the upper right part of abdomen that may extend into the back and shoulder, nausea and vomiting, general weakness and fatigue, an enlarged liver, abdominal swelling (ascites), and a yellow discoloration of the skin and the whites of eyes (jaundice). One skilled in oncology may readily identify symptoms associated with a particular cancer type.

In some embodiments, the subject is chemotherapy naïve.

In some embodiments, the subject has at least one EGFR-activating mutation.

EGFR activating mutations that may be associated with cancer include point mutations, deletion mutations, insertion mutations, inversions or gene amplifications that lead to an increase in at least one biological activity of EGFR, such as elevated tyrosine kinase activity, enhanced ligand binding, ligand-independent signaling, increased cell proliferation, signaling to MAPK/ERK pathways, gene transcription, formation of receptor homodimers and heterodimers, dimerization (EGFR:EGFR), heterodimerization (EGFR:HER2 or EGFR:HER3). Mutations can be located in any portion of an EGFR gene or regulatory region associated with an EGFR gene and include mutations in exon 18, 19, 20 or 21 or mutations in the kinase domain. Other examples of EGFR activating mutations are known in the art (see e.g., U.S. Pat. Publ. No. US2005/0272083, which is incorporated herein by reference in its entirety). Information about EGFR and other ErbB receptors including receptor homo- and hetero-dimers, receptor ligands, autophosphorylation sites, and signaling molecules involved in ErbB mediated signaling is known in the art (see e.g., Hynes and Lane, Nature Reviews Cancer 5: 341-354, 2005, which is incorporated herein by reference in its entirety).

In some embodiments, the EGFR activating mutation comprises G719A, G719X (X being any amino acid), L861X (X being any amino acid), L858R, E746K, L747S, E749Q, A750P, A755V, V765M, L858P or T790M substitution, deletion of E746-A750, deletion of R748-P753, insertion of Ala (A) between M766 and A767, insertion of Ser, Val and Ala (SVA) between 5768 and V769, insertion of Asn and Ser (NS) between P772 and H773, insertion of one or more amino acids between D761 and E762, A763 and Y764, Y764 and Y765, M766 and A767, A767 and V768, S768 and V769, V769 and D770, D770 and N771, N771 and P772, P772 and H773, H773 and V774, V774 and C775, one or more deletions in EGFR exon 19, one or more deletions in EGFR exon 20, one or more insertions in EGFR exon 20, or any combination thereof.

In some embodiments, the EGFR activating mutation comprises one or more uncommon EGFR activating mutations such as 57681, L861Q and G719X (X being any amino acid).

In some embodiments, the EGFR-activating mutation may be selected from one or more deletions in exon 19, L858R, and T790M. In some embodiments, the EGFR-activating mutation is one or more deletions in exon 19. In some embodiments, the EGFR-activating mutation is L858R. In some embodiments, the EGFR-activating mutation is selected from one or more deletions in exon 19 and L858R. A 5 amino acid deletion in exon 19 or the point mutation L858R in EGFR may be associated with EGFR-TKI sensitivity (Nakata and Gotoh, Expert Opin Ther Targets 16:771-781, 2012, which is incorporated herein by reference in its entirety). In tumor models driven by TKI-sensitive EGFR mutations such as L858R or exon 19 deletions, amivantamab has several proposed mechanisms of action (MOAs) including blocking ligand binding, receptor downmodulation, downstream signaling inhibition and triggering immune-directed antitumor activity (Commins et al., J Allergy Clin Immun 2010; 125(2):553-572, which is incorporated herein by reference in its entirety).

In some embodiments, the exon 19 deletions include E746_A750del, L747_P753delinsS, E746_S752delinsV, L747_A750delinsP, L747_T751 deletion, E746_P753delinsVS, E746_T751delinsA, E746_T751delinsL, L747_E749 deletion, L747_K754delinsATSPE, L747_K754delinsSN, L747_S752del, L747-T751delinsP, and T751-1759delinsN.

The presence or absence of any of the mutations disclosed herein such as, but not limited to those listed in groups (1) and (2), may be detected using methods known in the art, such as for example Sanger sequencing, next-generation sequencing (NGS), whole exome sequencing (WES), RNA-Seq, fluorescent in situ hybridization, or immunohistochemistry.

In some embodiments, the presence or absence of one or more mutations in a biological sample disclosed herein may be detected using next-generation sequencing (NGS). Non-limiting examples of biological samples are blood sample, plasma sample, and tumor sample. Another non-limiting example of a biological sample is circulating tumor DNA (ctDNA) isolated from the blood or plasma sample. In such embodiments, the one or more mutations are selected from PIK3CA E545K, PIK3CA E542K/V, PIK3CA H1047R, PIK3CA amplification, mutations in one or more genes from the RAS/RAF/MEK pathway as described herein, mutations in one or more genes from the WNT/b-catenin pathway as described herein, KRAS G12V/C/D/X (X being any amino acid other than G, V, C and D), KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletions, PTEN N48K, CDKN2A G101W, CDKN2B mutations, ALK fusions, FGFR3-TACC3 fusion, TPM3-NTRK1 fusion, RET fusions, BRAF fusions, and other oncogenic fusion events, EGFR C797S, EGFR L792H, EGFR amplification, EGFR G796S, EGFR L718X (X being any amino acid), EGFR E709K, EGFR G724S, MET amplification, MET exon 14 skipping (METex14) mutations, and EGFR-activating mutations as described herein.

In some embodiments, the methods of the present disclosure may comprise determining the presence or absence of one or more mutations in tumor DNA (e.g., ctDNA) obtained from a subject. In some embodiments, the methods of the present disclosure may comprise determining the presence or absence of one or more mutations in ctDNA. In some embodiments, the tumor DNA (e.g., ctDNA) is present in a biological sample isolated from the subject. As a non-limiting example, the biological sample is a blood sample, a plasma sample or a tumor sample. In some embodiments, the tumor DNA (e.g., ctDNA) may be isolated from the biological sample prior to mutation identification. In some embodiments, any of the tumor DNA (e.g., ctDNA) obtained from any of various biological samples disclosed herein may be optionally purified from said biological samples.

In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12. In some embodiments, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

In some embodiments, the bispecific anti-EGFR/c-Met antibody is an IgG1 isotype.

In some embodiments, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments, the bispecific anti-EGFR/c-Met antibody comprises a biantennary glycan structure with a fucose content of about between 1% to about 15%. In some embodiments, the bispecific anti-EGFR/c-Met antibody comprises a biantennary glycan structure with a fucose content of about between 2% to about 14%. In some embodiments, the bispecific anti-EGFR/c-Met antibody comprises a biantennary glycan structure with a fucose content of about between 3% to about 13%. In some embodiments, the bispecific anti-EGFR/c-Met antibody comprises a biantennary glycan structure with a fucose content of about between 4% to about 12%. In some embodiments, the bispecific anti-EGFR/c-Met antibody comprises a biantennary glycan structure with a fucose content of about between 5% to about 11%. In some embodiments, the bispecific anti-EGFR/c-Met antibody comprises a biantennary glycan structure with a fucose content of about 1%. In some embodiments, the bispecific anti-EGFR/c-Met antibody comprises a biantennary glycan structure with a fucose content of about 2%. In some embodiments, the bispecific anti-EGFR/c-Met antibody comprises a biantennary glycan structure with a fucose content of about 3%. In some embodiments, the bispecific anti-EGFR/c-Met antibody comprises a biantennary glycan structure with a fucose content of about 4%. In some embodiments, the bispecific anti-EGFR/c-Met antibody comprises a biantennary glycan structure with a fucose content of about 5%. In some embodiments, the bispecific anti-EGFR/c-Met antibody comprises a biantennary glycan structure with a fucose content of about 6%. In some embodiments, the bispecific anti-EGFR/c-Met antibody comprises a biantennary glycan structure with a fucose content of about 7%. In some embodiments, the bispecific anti-EGFR/c-Met antibody comprises a biantennary glycan structure with a fucose content of about 8%. In some embodiments, the bispecific anti-EGFR/c-Met antibody comprises a biantennary glycan structure with a fucose content of about 9%. In some embodiments, the bispecific anti-EGFR/c-Met antibody comprises a biantennary glycan structure with a fucose content of about 10%. In some embodiments, the bispecific anti-EGFR/c-Met antibody comprises a biantennary glycan structure with a fucose content of about 11%. In some embodiments, the bispecific anti-EGFR/c-Met antibody comprises a biantennary glycan structure with a fucose content of about 12%. In some embodiments, the bispecific anti-EGFR/c-Met antibody comprises a biantennary glycan structure with a fucose content of about 13%. In some embodiments, the bispecific anti-EGFR/c-Met antibody comprises a biantennary glycan structure with a fucose content of about 14%. In some embodiments, the bispecific anti-EGFR/c-Met antibody comprises a biantennary glycan structure with a fucose content of about 15%.

In some embodiments, the bispecific anti-EGFR/c-Met antibody disclosed herein may be administered in combination with a tyrosine kinase inhibitor (TKI) such as, but not limited to an epidermal growth factor receptor (EGFR TKI). Non-limiting examples of TKI are erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib. In some embodiments, the bispecific anti-EGFR/c-Met antibody disclosed herein may be administered in combination with lazertinib.

### Therapeutic Methods

In one aspect, the present disclosure provides a method for treating a cancer in a subject in need thereof based on the biomarker strategies described herein.

In some embodiments, the treatment method comprises a) determining the presence of one or more mutations in tumor DNA obtained from the subject, wherein the one or more mutations are selected from mutations in one or more genes from the RAS/RAF/MEK pathway and PIK3CA; and b) (i) when tumor DNA from said subject has no said mutations, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI), or (ii) when tumor DNA from said subject has one or more said mutations, administering to the subject a cancer therapy which does not include the combination therapy used in (i).

In some embodiments, the one or more mutations are further selected from mutations in one or more genes from the WNT/b-catenin pathway.

The mutations associated with RAS/RAF/MEK pathway or WNT/b-catenin pathway, or mutations in PIK3CA include those pathogenic mutations known in the art.

In some embodiments, the mutations may be found in one or more genes from RAS/RAF/MEK pathway, such as but not limited to, FGFR3, KRAS, BRAF, ERBB2, ALK, NRAS, PDGFRA and/or RET.

In some embodiments, the mutations in one or more genes from RAS/RAF/MEK pathway may include, but are not limited to, FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions.

In some embodiments, the KRAS G12X mutations are KRAS G12D, KRAS G12A, KRAS G12C, and KRAS G12V.

In some embodiments, mutations in BRAF may include those described in R. Yaeger et al., Targeting Alterations in the RAF-MEK Pathway. Cancer Discov (2019) 9 (3): 329-341, which is incoprated herein by reference in its entirety, for example, V600E/K/D/R/M, P367L/S, G464V/E, L485W, N486_A489delinsK, N486_P490del, E586K, L597Q/R/SN, T599TT/TS, T5991/K, K601E/N/T, K601_S602delinsNT, BRAF kinase duplication, fusions of BRAF kinase domain, D287H, V459L, G466A/E/V, S467L, G469E, N581I/S/T, D594A/G/H/N, F595L, G596D/R.

In some embodiments, mutations in BRAF may include those described in H. Yang et al., New Horizons in KRAS-Mutant Lung Cancer: Dawn After Darkness. Front. Oncol., 25 Sep. 2019, which is incoprated herein by reference in its entirety, for example, E3K, G12CN/D/A/S/R/F, G13C/D/EN/R, V14I, Q61L/E/H/R, F61L, L19F, D33E, T58I, A59T, A146PN/T, C118S, A59 G60delinsGV.

In some embodiments of the diagnostic or treatment method described above, wherein the mutations in PIK3CA comprise PIK3CA E545K.

In some embodiments, the mutations may be found in one or more genes from WNT/b-catenin pathway, such as but not limited to, APC and CTNNB1.

In some embodiments, the mutations in one or more genes from WNT/b-catenin pathway comprise APC Q1469, APC R405, APC 5713, CTNNB1 S33P, CTNNB1 S37C, CTNNB1 S37F, and CTNNB1 S45P.

In some embodiments, the treatment method comprises a) determining the presence of one or more mutations in tumor DNA (e.g., circulating tumor DNA (ctDNA)) obtained from the subject, wherein the one or more mutations are selected from the following two groups: (1) PIK3CA E545K, PIK3CA E542K/V, PIK3CA H1047R, PIK3CA amplification, KRAS G12V/C/D/X, KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletion, PTEN N48K, CDKN2A G101W, CDKN2B, ALK fusions, FGFR3-TACC3 and other fusions, RET fusions, BRAF fusions, and other oncogenic fusion events; (2) EGFR C797S, EGFR L792H, EGFR amplification, EGFR G796S, EGFR L718X (X being any amino acid), EGFR E709K, EGFR G724S, MET amplification, and MET exon 14 skipping (METex14) mutations; and b) (i) when tumor DNA (e.g., ctDNA) from said subject has either no mutations from group (1), or has one or more mutations from group (1) and one or more mutations from group (2), administering to the subject an effective amount of a combination therapy comprising a bispecific anti-epidermal growth factor receptor (EGFR)/hepatocyte growth factor receptor (c-Met) bispecific antibody and an EGFR tyrosine kinase inhibitor (TKI), or (ii) when tumor DNA (e.g., ctDNA) from said subject has one or more mutations from group (1) and no mutations from group (2), administering to the subject a cancer therapy which does not include the combination therapy used in (i).

Non-limiting examples of HER2 oncogenic alterations include HER2 Y772_A775 duplication, HER2 L755M/S/W and HER2 S310F/Y. Non-limiting examples of PTEN deletions include PTEN I33del and PTEN I14del. Non-limiting examples of ALK fusions include SQSTM1-ALK fusion and EML4-ALK fusion. Non-limiting examples of RET fusions include CCDC6-RET fusion, KIF5B-RET fusion, and NCOA4-RET fusion. Non-limiting examples of BRAF fusions include those described in Ross et al., Int. J. Cancer: 138, 881-890 (2016), which is incorporated herein by reference in its entirety, such as KIAA1549-BRAF, MKRN1-BRAF, TRIM24-BRAF, AGAP3-BRAF, ZC3HAV1-BRAF, AKAP9-BRAF, CCDC6-BRAF, AGK-BRAF, EPS15-BRAF, NUP214-BRAF, ARMC10-BRAF, BTF3L4-BRAF, GHR-BRAF, ZNF767-BRAF, CCDC91-BRAF, DYNC1I2-BRAF, ZKSCAN1-BRAF, GTF2I-BRAF, MZT1-BRAF, RAD18-BRAF, CUX1-BRAF, SLC12A7-BRAF, MYRIP-BRAF, SND1-BRAF, NUB1-BRAF, KLHL7-BRAF, TANK-BRAF, RBMS3-BRAF, STRN3-BRAF, STK35-BRAF, ETFA-BRAF, SVOPL-BRAF, and JHDM1D-BRAF. Other oncogenic fusion events include, but are not limited to, those disclosed in FIG. 1 of Gao et al., Cell Rep. 2018 Apr. 3; 23(1): 227-238.e3., which is incorporated herein by reference in its entirety.

In another aspect, the present disclosure provides a method for treating a cancer in a subject in need thereof, said method comprising a) determining the expression level of EGFR and MET in a tumor sample obtained from the subject using immunohistochemistry (IHC), b) calculating a combined H score based on the expression level of EGFR and MET determined in step (a), and c) (i) when the combined H score is greater than or equal to 400, administering to the subject an effective amount of a combination therapy comprising a bispecific anti-epidermal growth factor receptor (EGFR)/hepatocyte growth factor receptor (c-Met) bispecific antibody and an EGFR tyrosine kinase inhibitor (TKI); (ii) when the combined H score is less than 400, not administering to the subject the combination therapy used in (i) or administering to the subject a cancer therapy which does not include the combination therapy used in (i).

In another aspect, the present disclosure provides a method for treating a cancer in a subject in need thereof, said method comprising a) determining the expression level of EGFR or MET in a tumor sample obtained from the subject using immunohistochemistry (IHC), b) determining a staining intensity score, on a scale from 0 to 3+, based on the expression level of EGFR or MET determined in step (a), and c) (i) when the staining intensity score is 3+, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI); or (ii) when the staining intensity score is less than 3+, not administering to the subject the combination therapy used in (i) or administering to the subject a cancer therapy which does not include the combination therapy used in (i).

In some embodiments, the method comprises in step (c), (i) when the staining intensity score is 3+ in greater than or equal to 5%, 7.5%, 10%, 12.5%, 15%, 17.5%, 20%, 22.5%, 25%, 27.5%, 30%, 32.5%, 35%, 37.5%, 40%, 42.5%, 45%, 47.5% or 50% of the cells of the tumor sample, administering to the subject a therapeutically effective amount of the combination therapy; or (ii) when the staining intensity score is 3+ in less than 5%, 7.5%, 10%, 12.5%, 15%, 17.5%, 20%, 22.5%, 25%, 27.5%, 30%, 32.5%, 35%, 37.5%, 40%, 42.5%, 45%, 47.5% or 50% cells of the tumor sample, not administering to the subject the combination therapy used in (i) or administering to the subject a cancer therapy which does not include the combination therapy used in (i).

In one embodiment, the method comprises in step (c), (i) when the staining intensity score is 3+ in greater than or equal to 25% of the cells of the tumor sample, administering to the subject a therapeutically effective amount of the combination therapy; or (ii) when the staining intensity score is 3+ in less 25% cells of the tumor sample, not administering to the subject the combination therapy used in (i) or administering to the subject a cancer therapy which does not include the combination therapy used in (i).

In various embodiments, the cancer is a solid tumor, a brain tumor, or a hematologic malignancy. In certain embodiments, the hematologic malignancy is AML, ALL, B-ALL, T-ALL, or lymphoma. Examples of tumors are, but not limited to, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., Ewing sarcoma and other Ewing sarcoma family of tumors, osteosarcoma, or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In various embodiments, the tumor is selected from osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma. In various embodiments, the tumor is a solid tumor. In various embodiments, the solid tumor is Ewings sarcoma, lung adenocarcinoma, osteosarcoma, breast cancer, or prostate cancer. In certain embodiments, the tumor is a brain tumor. In some embodiments, the brain tumor is glioblastoma or neuroblastoma.

In some embodiments, methods of the present disclosure may be useful for treating cancer selected from squamous cell cancer, adenosquamous cell carcinoma, lung cancer, cancer of the peritoneum, hepatocellular cancer, gastric or stomach cancer, cervical cancer, ovarian cancer, liver cancer, bladder cancer, cancer of the urinary tract, hepatoma, breast cancer, colon cancer, colorectal cancer, endometrial cancer, salivary gland carcinoma, kidney or renal cancer, prostate cancer, vulval cancer, thyroid cancer, hepatic carcinoma, anal carcinoma, penile carcinoma, skin cancer, multiple myeloma and acute lymphocytic leukemia (ALL), acute myelocytic leukemia (AML), chronic myelocytic leukemia (CML), and chronic lymphocytic leukemia (CLL), lymphoma such as Hodgkin lymphoma (HL) and non-Hodgkin lymphoma (NHL), follicular lymphoma, chronic lymphocytic leukemia/small lymphocytic lymphoma (CLL/SLL), mantle cell lymphoma (MCL), marginal zone B-cell lymphomas, primary mediastinal B-cell lymphoma, Burkitt lymphoma, lymphoplasmacytic lymphoma, immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL), angioimmunoblastic T-cell lymphoma, extra-nodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, brain, as well as head and neck cancer, biliary cancer, bronchus cancer, chordoma, choriocarcinoma, epithelial carcinoma, endothelial sarcoma, esophageal cancer, Ewing sarcoma, heavy chain disease, hematopoietic cancer, immunocytic amyloidosis, monoclonal gammopathy of undetermined significance, myelodysplastic syndromes, myeloproliferative disorder, agnogenic myeloid metaplasia (AMM) or myelofibrosis (MF), chronic idiopathic myelofibrosis, myeloproliferative neoplasms, polycythemia vera, rectum adenocarcinoma, essential thrombocytosis, chronic neutrophilic leukemia, hypereosinophilic syndrome, or soft tissue sarcoma, and metastases thereof.

In some embodiments, methods of the present disclosure may be useful for treating lung cancer. In some embodiments, the lung cancer is non-small cell lung cancer (NSCLC).

In some embodiments, methods of the present disclosure may be useful for treating a cancer in a subject in need thereof, wherein the subject is relapsed or resistant to treatment with one or more prior anti-cancer therapies. In some embodiments, the one or more prior anti-cancer therapies comprises one or more EGFR TKIs, wherein the EGFR TKIs are not the same as the EGFR TKI used in the combination therapy of the present disclosure. In some embodiments, the one or more EGFR TKIs comprises osimertinib, erlotinib, afatinib, rociletinib, olmutinib, or any combination thereof.

In some embodiments, the methods of the present disclosure useful for treating a cancer in a subject in need thereof may comprise administering to a subject an effective amount of a combination therapy comprising a bispecific anti-epidermal growth factor receptor (EGFR)/hepatocyte growth factor receptor (c-Met) bispecific antibody and an EGFR tyrosine kinase inhibitor (TKI). In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12. In some embodiments, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16. In some embodiments, the bispecific anti-EGFR/c-Met antibody is an IgG1 isotype. In some embodiments, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20. In some embodiments, the bispecific anti-EGFR/c-Met antibody comprises a biantennary glycan structure with a fucose content of about between 1% to about 15%.

In some embodiments, the bispecific anti-EGFR/c-Met antibody disclosed herein may be administered in combination with a tyrosine kinase inhibitor (TKI) such as, but not limited to an epidermal growth factor receptor (EGFR TKI). Non-limiting examples of TKI are erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib. In some embodiments, the bispecific anti-EGFR/c-Met antibody disclosed herein may be administered in combination with lazertinib.

In some embodiments, the methods of the present disclosure may be useful for treating a cancer in a subject in need thereof, wherein said methods comprise administering to a subject a cancer therapy which does not include the combination therapy comprising a bispecific anti-EGFR/c-Met bispecific antibody and an EGFR TKI disclosed herein.

In some embodiments, the one or more cancer therapies comprises one or more chemotherapeutic agents, checkpoint inhibitors, targeted cancer therapies or kinase inhibitors, or any combination thereof.

In some embodiments, the kinase inhibitor is an inhibitor of EGFR, an inhibitor of MET, an inhibitor of HER2, an inhibitor of HER3, an inhibitor of HER4, an inhibitor of VEGFR or an inhibitor of AXL. In some embodiments, the kinase inhibitor is an inhibitor of EGFR. In some embodiments, the kinase inhibitor is an inhibitor of MET. In some embodiments, the kinase inhibitor is an inhibitor of HER2. In some embodiments, the kinase inhibitor is an inhibitor of HER3. In some embodiments, the kinase inhibitor is an inhibitor of HER4. In some embodiments, the kinase inhibitor is an inhibitor of VEGFR. In some embodiments, the kinase inhibitor is an inhibitor of AXL.

In some embodiments, the one or more cancer therapies comprises carboplatin, paclitaxel, gemcitabine, cisplatin, vinorelbine, docetaxel, palbociclib, crizotinib, PD-(L)1 axis inhibitor, an inhibitor of EGFR, an inhibitor of MET, an inhibitor of HER2, an inhibitor of HER3, an inhibitor of HER4, an inhibitor of VEGFR, an inhibitor of AXL, erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib, or any combination thereof. Exemplary PD-(L)1 axis inhibitors are antibodies that bind PD-1 such as nivolumab (OPDIVO®), pembrolimumab (KEYTRUDA®), sintilimab, cemiplimab (LIBTAYO®), tripolibamab, tislelizumab, spartalizumab, camrelizumab, dostralimab, genolimzumab or cetrelimab, or antibodies that bind PD-L1, such as PD-L1 antibodies are envafolimab, atezolizumab (TECENTRIQ®), durvalumab (IMFINZI®) and avelumab (BAVENCIO®). Marketed antibodies may be purchased via authorized distributor or pharmacy. The amino acid sequences structures of the small molecules can be found from USAN and/or INN submissions by the companies of from CAS registry.

In some embodiments, the cancer therapy which does not include the combination therapy of the present disclosure may be a platinum-based chemotherapy such as but not limited to carboplatin, cisplatin, or a combination thereof.

In some embodiments, methods of the present disclosure may be useful for treating a cancer in a subject in need thereof, wherein the subject is chemotherapy naïve.

In some embodiments, methods of the present disclosure may be useful for treating a cancer in a subject in need thereof, wherein the subject has at least one EGFR-activating mutation. Non-limiting examples of EGFR-activating mutations are exon 19 deletions, L858R, and T790M.

### Administration

The bispecific anti-EGFR/c-Met antibody may be administered in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used to formulate the bispecific anti-EGFR/c-Met antibody. These solutions are sterile and generally free of particulate matter. They may be sterilized by conventional, well-known sterilization techniques (e.g., filtration). For parenteral administration, the carrier may comprise sterile water and other excipients may be added to increase solubility or preservation. Injectable suspensions or solutions may also be prepared utilizing aqueous carriers along with appropriate additives. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, Pa. 2006, Part 5, Pharmaceutical Manufacturing pp 691-1092, See especially pp. 958-989.

The mode of administration may be any suitable route that delivers the bispecific anti-EGFR-c-Met antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intratumoral, intra-articular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

In some embodiments, the bispecific anti-EGFR/c-Met antibody is administered intravenously.

In some embodiments, the bispecific anti-EGFR/c-Met antibody is administered subcutaneously or intradermally to the subject. The bispecific anti-EGFR/c-Met antibody may be administered subcutaneously or intradermally at a dose sufficient to achieve a therapeutic effect in the subject.

In some embodiments, the bispecific anti-EGFR/c-Met antibody is administered at a dose of between about 140 mg to about 2240 mg. In some embodiments, the bispecific anti-EGFR/c-Met antibody is administered at a dose of between about 140 mg to about 1750 mg.

In some embodiments, the bispecific anti-EGFR/c-Met antibody is administered at a dose of about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, 1575 mg, about 1580 mg, about 1590 mg, about 1600 mg, about 1610 mg, 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, 1890 mg, about 1900 mg, about 1910 mg, about 1920 mg, about 1930 mg, about 1940 mg, about 1950 mg, about 1960 mg, about 1970 mg, about 1980 mg, about 1990 mg, about 2000 mg, 2100 mg, 2110 mg, 2120 mg, 2130 mg, 2140 mg, 2150 mg, 2160 mg, 2170 mg, 2180 mg, 2190 mg, 2200 mg, 2210 mg, 2220 mg, 2230 mg, 2240 mg, 2250 mg, or 2260 mg.

In some embodiments, the bispecific anti-EGFR/c-Met antibody is administered at a dose of about 350 mg, about 700 mg, about 1050 mg or about 1400 mg. In some embodiments, the bispecific anti-EGFR/c-Met antibody is administered at a dose of about 350 mg. In some embodiments, the bispecific anti-EGFR/c-Met antibody is administered at a dose of about 700 mg. In some embodiments, the bispecific anti-EGFR/c-Met antibody is administered at a dose of about 750 mg. In some embodiments, the bispecific anti-EGFR/c-Met antibody is administered at a dose of about 800 mg. In some embodiments, the bispecific anti-EGFR/c-Met antibody is administered at a dose of about 850 mg. In some embodiments, the bispecific anti-EGFR/c-Met antibody is administered at a dose of about 900 mg. In some embodiments, the bispecific anti-EGFR/c-Met antibody is administered at a dose of about 950 mg. In some embodiments, the bispecific anti-EGFR/c-Met antibody is administered at a dose of about 1000 mg. In some embodiments, the bispecific anti-EGFR/c-Met antibody is administered at a dose of about 1050 mg. In some embodiments, the bispecific anti-EGFR/c-Met antibody is administered at a dose of about 1100 mg. In some embodiments, the bispecific anti-EGFR/c-Met antibody is administered at a dose of about 1150 mg. In some embodiments, the bispecific anti-EGFR/c-Met antibody is administered at a dose of about 1200 mg. In some embodiments, the bispecific anti-EGFR/c-Met antibody is administered at a dose of about 1250 mg. In some embodiments, the bispecific anti-EGFR/c-Met antibody is administered at a dose of about 1300 mg. In some embodiments, the bispecific anti-EGFR/c-Met antibody is administered at a dose of about 1350 mg. In some embodiments, the bispecific anti-EGFR/c-Met antibody is administered at a dose of about 1400 mg.

In some embodiments, the bispecific anti-EGFR/c-Met antibody is administered is administered at a dose of 1050 mg if the subject has a body weight of less than 80 kg. In some embodiments, the bispecific anti-EGFR/c-Met antibody is administered at a dose of 1400 mg if the subject has a body weight of greater than or equal to 80 kg.

In some embodiments, the bispecific anti-EGFR/c-Met antibody is administered once a week. In some embodiments, the bispecific anti-EGFR/c-Met antibody is administered about 1050 mg once a week. In some embodiments, the bispecific anti-EGFR/c-Met antibody is administered about 1400 mg once a week.

In some embodiments, the bispecific anti-EGFR/c-Met antibody is administered once in two weeks. In some embodiments, the bispecific anti-EGFR/c-Met antibody is administered about 1050 mg once in two weeks. In some embodiments, the bispecific anti-EGFR/c-Met antibody is administered about 1400 mg once in two weeks.

In some embodiments, the bispecific anti-EGFR/c-Met antibody is administered twice a week. In some embodiments, the bispecific anti-EGFR/c-Met antibody is administered once a week. In some embodiments, the bispecific anti-EGFR/c-Met antibody is administered once in two weeks. In some embodiments, the bispecific anti-EGFR/c-Met antibody is administered once in three weeks. In some embodiments, the bispecific anti-EGFR/c-Met antibody is administered once in four weeks.

In some embodiments, the bispecific anti-EGFR/c-Met antibody is administered twice a week, once a week, once in two weeks, once in three weeks or once in four weeks.

In some embodiments, the bispecific anti-EGFR/c-Met antibody disclosed herein may be administered in combination with a tyrosine kinase inhibitor TKI such as, but not limited to an epidermal growth factor receptor (EGFR TKI). Non-limiting examples of TKI are erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib. In some embodiments, the EGFR TKI is lazertinib.

For combination therapies, the EGFR TKI may be administered using recommended doses and dosages of the EGFR TKI.

The mode of administration may be any suitable route that delivers EGFR TKI to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intratumoral, intra-articular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

In some embodiments, the mode of administration that may the suitable route that delivers lazertinib to the subject may be oral administration.

In some embodiments, the EGFR TKI is administered at a dose of between about 10 mg to about 400 mg. In some embodiments, the EGFR TKI is administered at a dose of between about 20 mg to about 320 mg. In some embodiments, the EGFR TKI is administered at a dose of between about 50 mg to about 300 mg. In some embodiments, the EGFR TKI is administered at a dose of between about 100 mg to about 300 mg. In some embodiments, the EGFR TKI is administered at a dose of between about 150 mg to about 280 mg. In some embodiments, the EGFR TKI is administered at a dose of between about 200 mg to about 250 mg. In some embodiments, the EGFR TKI is administered at a dose of between about 220 mg to about 250 mg.

In some embodiments, the EGFR TKI is administered at a dose of about 20 mg, about 50 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg. In some embodiments, the EGFR TKI is administered at a dose of about 240 mg.

In some embodiments, the EGFR TKI is administered daily. In some embodiments, the EGFR TKI is administered twice a week. In some embodiments, the EGFR TKI is administered once a week. In some embodiments, lazertinib is administered once in two weeks. In some embodiments, lazertinib is administered once in three weeks. In some embodiments, the EGFR TKI is administered once in four weeks.

In some embodiments, the bispecific anti-EGFR/c-Met antibody disclosed herein may be administered in combination with lazertinib, which may be administered using any of the doses and dosages disclosed herein. In some embodiments, lazertinib is administered at a dose of between about 10 mg to about 400 mg. In some embodiments, lazertinib is administered at a dose of between about 20 mg to about 320 mg. In some embodiments, lazertinib is administered at a dose of about 20 mg, about 50 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg. In some embodiments, lazertinib is administered at a dose of about 240 mg.

In some embodiments, the bispecific anti-EGFR/c-Met antibody disclosed herein may be administered in any of these doses and dosages disclosed herein in combination with lazertinib, which may be administered in any of these doses and dosages disclosed herein. As a non-limiting example, 700 mg amivantamab may be administered in combination with 240 mg lazertinib. As a non-limiting example, 1050 mg amivantamab may be administered in combination with 240 mg lazertinib. As a non-limiting example, 1050 mg amivantamab may be administered in combination with 240 mg lazertinib. As a non-limiting example, 1400 mg amivantamab may be administered in combination with 240 mg lazertinib.

In some embodiments, the bispecific anti-EGFR/c-Met antibody disclosed herein may be administered in combination with lazertinib, wherein lazertinib is administered daily, every other day, twice a week, or once a week. In some embodiments, the bispecific anti-EGFR/c-Met antibody disclosed herein may be administered in combination with lazertinib, wherein lazertinib is administered daily. In some embodiments, the bispecific anti-EGFR/c-Met antibody disclosed herein may be administered in combination with lazertinib, wherein lazertinib is administered orally.

In some embodiments, the combination therapy comprising a bispecific anti-EGFR/c-Met bispecific antibody and an EGFR TKI may further include one or more additional anti-cancer therapies.

In some embodiments, the methods of the present disclosure comprise administering to a subject a cancer therapy which does not include the combination therapy comprising a bispecific anti-EGFR/c-Met bispecific antibody and an EGFR TKI disclosed herein. In some embodiments, the cancer therapy may include any one of those described herein. As a non-limiting example, the cancer therapy that may be administered in the methods of the disclosure may comprise any number of various platinum-based chemotherapies or combinations thereof. As a non-limiting example, the platinum-based chemotherapy comprises carboplatin, cisplatin, or a combination thereof.

Additional anti-cancer therapies that may be administered in the methods of the disclosure may include any one or more of the chemotherapeutic drugs or other anti-cancer therapeutics known to those of skill in the art. Chemotherapeutic agents are chemical compounds useful in the treatment of cancer and include growth inhibitory agents or other cytotoxic agents and include alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL®docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA®), afatinib (BIBW 2992), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et aL, eds.), Chapter 10, McMillan Publishing, are also applicable to the methods of the present invention.

### Generation of Bispecific Anti-EGFR/c-Met Antibodies Used in the Methods of the Disclosure

An exemplary bispecific anti-EGFR/c-Met antibody that can be used in the methods of the disclosures is amivantamab. Amivantamab or JNJ-61186372 (JNJ-372) is an IgG1 anti-EGFR/c-Met bispecific antibody described in U.S. Pat. No. 9,593,164, which is incorporated herein by reference in its entirety. Amivantamab is characterized by following amino acid sequences:

EGFR binding arm
(HCDR1, EGFR binding arm)
>SEQ ID NO: 1
TYGMH
(HCDR2, EGFR binding arm)
>SEQ ID NO: 2
VIWDDGSYKYYGDSVKG
(HCDR3, EGFR binding arm)
>SEQ ID NO: 3
DGITMVRGVMKDYFDY
(LCDR1, EGFR binding arm)
>SEQ ID NO: 4
RASQDISSALV
(LCDR2, EGFR binding arm)
>SEQ ID NO: 5
DASSLES
(LCDR3, EGFR binding arm)
>SEQ ID NO: 6
QQFNSYPLT
(HCDR1, c-Met binding arm)
>SEQ ID NO: 7
SYGIS
(HCDR2, c-Met binding arm)
>SEQ ID NO: 8
WISAYNGYTNYAQKLQG
(HCDR3, c-Met binding arm)
>SEQ ID NO: 9
DLRGTNYFDY
(LCDR1, c-Met binding arm)
>SEQ ID NO: 10
RASQGISNWLA
(LCDR2, c-Met binding arm)
>SEQ ID NO: 11
AASSLLS
(LCDR3, c-Met binding arm)
>SEQ ID NO: 12
QQANSFPIT
(VH, EGFR binding arm)
>SEQ ID NO: 13
QVQLVESGGGVVQPGRSLRLSCAASGFTFSTYGMHWVRQAPGKGLEWVAVI
WDDGSYKYYGDSVKGRFTISRDNSKNTLYLQMNSLRAEDTAVYYCARDGIT
MVRGVMKDYFDYWGQGTLVTVSS
(VL, EGFR binding arm)
>SEQ ID NO: 14
AIQLTQSPSSLSASVGDRVTITCRASQDISSALVWYQQKPGKAPKLLIYDA
SSLESGVPSRFSGSESGTDFTLTISSLQPEDFATYYCQQFNSYPLTFGGGT
KVEIK
(VH, c-Met binding arm)
>SEQ ID NO: 15
QVQLVQSGAEVKKPGASVKVSCETSGYTFTSYGISWVRQAPGHGLEWMGWI
SAYNGYTNYAQKLQGRVTMTTDTSTSTAYMELRSLRSDDTAVYYCARDLRG
TNYFDYWGQGTLVTVSS
(VL, c-Met binding arm)
>SEQ ID NO: 16
DIQMTQSPSSVSASVGDRVTITCRASQGISNWLAWFQHKPGKAPKLLIYAA
SSLLSGVPSRFSGSGSGTDFTLTISSLQPEDFATYYCQQANSFPITFGQGT
RLEIK
HC1
>SEQ ID NO: 17
QVQLVESGGGVVQPGRSLRLSCAASGFTFSTYGMHWVRQAPGKGLEWVAVI
WDDGSYKYYGDSVKGRFTISRDNSKNTLYLQMNSLRAEDTAVYYCARDGIT
MVRGVMKDYFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDS
DGSFLLYSKLTVDKSRWQQGNVFSCSVMHEALHNHYTQKSLSLSPGK
LC1
>SEQ ID NO: 18
AIQLTQSPSSLSASVGDRVTITCRASQDISSALVWYQQKPGKAPKLLIYDA
SSLESGVPSRFSGSESGTDFTLTISSLQPEDFATYYCQQFNSYPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC
HC2
>SEQ ID NO: 19
QVQLVQSGAEVKKPGASVKVSCETSGYTFTSYGISWVRQAPGHGLEWMGWI
SAYNGYTNYAQKLQGRVTMTTDTSTSTAYMELRSLRSDDTAVYYCARDLRG
TNYFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNV
NHKPSNTKVDKRVEPKSCDKTHTCPPCPAPELLGGPSVFLFPPKPKDTLMI
SRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFL
YSRLTVDKSRWQQGNVFSCSVMHEALHNHYTQKSLSLSPGK
LC2
>SEQ ID NO: 20
DIQMTQSPSSVSASVGDRVTITCRASQGISNWLAWFQHKPGKAPKLLIYAA
SSLLSGVPSRFSGSGSGTDFTLTISSLQPEDFATYYCQQANSFPITFGQGT
RLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC

In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In some embodiments, the first domain that specifically binds EGFR comprises a heavy chain variable region (VH) of SEQ ID NO: 13 and a light chain variable region (VL) of SEQ ID NO: 14; and the second domain that specifically binds c-Met comprises the VH of SEQ ID NO: 15 and the VL of SEQ ID NO: 16.

In some embodiments, the bispecific anti-EGFR/c-Met antibody is an IgG1 isotype.

In some embodiments, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In one embodiment, the bispecific anti-EGFR/c-Met antibody comprises one or more Fc silencing mutations.

In one embodiment, the one or more Fc silencing mutations decrease affinity to Fcγ receptors.

In one embodiment, the one or more Fc silencing mutations comprise V234A/G237A/P238S/H268A/V309L/A330S/P331S.

In one embodiment, the bispecific anti-EGFR/c-Met antibody comprises a biantennary glycan structure with a fucose content between about 1% to about 15%. Antibodies with reduced fucose content can be made using different methods reported to lead to the successful expression of relatively high defucosylated antibodies bearing the biantennary complex-type of Fc oligosaccharides such as control of culture osmolality (Konno et al., Cytotechnology 64(:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the □ 1,6-fucosyltrasferase (FUT8) gene (Mori et al., Biotechnol Bioeng 88:901-908, 2004), or coexpression of β-1,4-N-acetylglucosaminyltransferase III and Golgi a-mannosidase II or a potent alpha-mannosidase I inhibitor, kifunensine (Ferrara et al., J Biol Chem281:5032-5036, 2006, Ferrara et al., Biotechnol Bioeng 93:851-861, 2006; Xhou et al., Biotechnol Bioeng 99:652-65, 2008). In general, lowering fucose content in the glycan of the antibodies potentiates antibody-mediated cellular cytotoxicity (ADCC).

Other bispecific anti-EGFR/c-Met antibodies publicly available may also be used in the methods of the disclosure as long as they demonstrate similar characteristics when compared to amivantamab as described in U.S. Pat. No. 9,593,164. Bispecific anti-EGFR/c-Met antibodies that may be used in the methods of the disclosure may also be generated by combining EGFR binding VH/VL domains and c-Met binding VH/VL domains that are publicly available and testing the resulting bispecific antibodies for their characteristics as described in U.S. Pat. No. 9,593,164.

Bispecific anti-EGFR/c-Met antibodies used in the methods of the disclosure may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e., an epitope on EGFR and an epitope on c-Met. For example, the bispecific antibodies of the invention may be generated using the technology described in Int. Pat. Publ. No. WO2011/131746. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. For IgG4 antibodies, a wild-type IgG4 and a IgG4 antibody with F405L and R409K substitutions may be used. To generate bispecific antibodies, first monospecific bivalent antibody and the second monospecific bivalent antibody are engineered to have the aforementioned mutation in the Fc region, the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Bispecific anti-EGFR/c-Met antibodies used in the methods of the disclosure may also be generated using designs such as the Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody)(EMD Serono), and the Biclonic (Merus).

In the “knob-in-hole” strategy (see, e.g., Intl. Publ. No. WO 2006/028936) select amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S L368A_Y407V.

CrossMAb technology, in addition to utilizing the “knob-in-hole” strategy to promoter Fab arm exchange utilizes CH1/CL domain swaps in one half arm to ensure correct light chain pairing of the resulting bispecific antibody (see e.g., U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by following substitutions (expressed as modified positions in the first CH3 domain of the first heavy chain/modified position in the second CH3 domain of the second heavy chain): L351Y_F405A_Y407V/T394W, T366_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Patent Publ. No. US2012/0149876 or U.S. Patent Publ. No. US2013/0195849.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

### Kits

The invention also provides a kit comprising any of one or more reagents for determining the presence or level of one or more biomarkers described herein. The kit may be used for therapeutic uses and/or as diagnostic kits.

The kit may include one or more other elements including: packaging; instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit of the present disclosure comprises one or more reagents for determining the presence of any one or more mutations described herein such as, but not limited to mutations in tumor DNA from a subject with a cancer, e.g., a lung cancer. In some embodiments, the tumor DNA is circulating tumor DNA (ctDNA).

The kit may be used to detect the presence of a mutation. Non-limiting examples of mutations are PIK3CA E545K, PIK3CA E542K/V, PIK3CA H1047R, PIK3CA amplification, mutations in one or more genes from the RAS/RAF/MEK pathway as described herein, mutations in one or more genes from the WNT/b-catenin pathway as described herein, KRAS G12V/C/D/X (X being any amino acid other than G, V, C and D), KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletion, PTEN N48K, CDKN2A G101W, CDKN2B mutations, ALK fusions, FGFR3-TACC3 and other fusions (e.g., TPM3-NTRK1 fusion), RET fusions, BRAF fusions, and other oncogenic fusion events, EGFR C797S, EGFR L792H, EGFR amplification, EGFR G796S, EGFR L718X (X being any amino acid), EGFR E709K, EGFR G724S, MET amplification, and MET exon 14 skipping (METex14) mutations.

In some embodiments, the mutations in one or more genes from RAS/RAF/MEK pathway comprise FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions. In some embodiments, the KRAS G12X mutations are KRAS G12D, KRAS G12A, KRAS G12C, and KRAS G12V.

In some embodiments, the mutations in one or more genes from WNT/b-catenin pathway comprise APC Q1469, APC R405, APC 5713, CTNNB1 S33P, CTNNB1 S37C, CTNNB1 S37F, and CTNNB1 S45P.

Non-limiting examples of HER2 oncogenic alterations include HER2 Y772_A775 duplication, HER2 L755M/S/W and HER2 S310F/Y. Non-limiting examples of PTEN deletions include PTEN I33del and PTEN4del. Non-limiting examples of ALK fusions include SQSTM1-ALK fusion and EML4-ALK fusion. Non-limiting examples of RET fusions include CCDC6-RET fusion, KIF5B-RET fusion, and NCOA4-RET fusion. Non-limiting examples of BRAF fusions include those described in Ross et al., Int. J. Cancer: 138, 881-890 (2016), which is incorporated herein by reference in its entirety, such as KIAA1549-BRAF, MKRN1-BRAF, TRIM24-BRAF, AGAP3-BRAF, ZC3HAV1-BRAF, AKAP9-BRAF, CCDC6-BRAF, AGK-BRAF, EPS15-BRAF, NUP214-BRAF, ARMC10-BRAF, BTF3L4-BRAF, GHR-BRAF, ZNF767-BRAF, CCDC91-BRAF, DYNC1I2-BRAF, ZKSCAN1-BRAF, GTF2I-BRAF, MZT1-BRAF, RAD18-BRAF, CUX1-BRAF, SLC12A7-BRAF, MYRIP-BRAF, SND1-BRAF, NUB1-BRAF, KLHL7-BRAF, TANK-BRAF, RBMS3-BRAF, STRN3-BRAF, STK35-BRAF, ETFA-BRAF, SVOPL-BRAF, and JHDM1D-BRAF. Other oncogenic fusion events include, but are not limited to, those disclosed in FIG. 1 of Gao et al., Cell Rep. 2018 Apr. 3; 23(1): 227-238.e3., which is incorporated herein by reference in its entirety.

In another aspect, provided herein is a diagnostic kit comprising (i) one or more reagents for determining the presence of one or more mutations in tumor DNA from a subject with a cancer, and (ii) optionally packaging and/or instructions for use, wherein the one or more mutations are selected from mutations in one or more genes from the RAS/RAF/MEK pathway and mutations in PIK3CA. In some embodiments, the one or more genes from RAS/RAF/MEK pathway are FGFR3, KRAS, BRAF, ERBB2, ALK, NRAS, PDGFRA and/or RET. In some embodiments, the mutations in one or more genes from RAS/RAF/MEK pathway comprise FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions. In some embodiments, the KRAS G12X mutations are KRAS G12D, KRAS G12A, KRAS G12C, and KRAS G12V. In some embodiments, the mutations in PIK3CA comprise PIK3CA E545K.

In some embodiments of the kit described above, the one or more mutations are further selected from mutations in one or more genes from the WNT/b-catenin pathway. In some embodiments, the one or more genes from WNT/b-catenin pathway are APC and CTNNB1. In some embodiments, the mutations in one or more genes from WNT/b-catenin pathway comprise APC Q1469, APC R405, APC S713, CTNNB1 S33P, CTNNB1 S37C, CTNNB1 S37F, and CTNNB1 S45P.

In some aspects, the kit may be a diagnostic kit, wherein the diagnostic kit comprises (i) one or more reagents for determining the presence of one or more mutations in tumor DNA (e.g., circulating tumor DNA (ctDNA)) from a subject with a cancer, and (ii) optionally packaging and/or instructions for use, wherein the one or more mutations are selected from the following two groups: (1) PIK3CA E545K, PIK3CA E542K/V, PIK3CA H1047R, PIK3CA amplification, KRAS G12V/C/D/X (X being any amino acid other than G, V, C and D), KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletion, PTEN N48K, CDKN2A G101W, CDKN2B mutations, ALK fusions, FGFR3-TACC3 and other fusions (e.g., TPM3-NTRK1 fusion), RET fusions, BRAF fusions, and other oncogenic fusion events; and (2) EGFR C797S, EGFR L792H, EGFR amplification, EGFR G796S, EGFR L718X (X being any amino acid), EGFR E709K, EGFR G724S, MET amplification, and MET exon 14 skipping (METex14) mutations. In some embodiments, the ctDNA may be present in a biological sample isolated from a subject. The biological sample may be any of the biological samples of the present disclosure such as, but not limited to a blood sample or a plasma sample. In some embodiments the tumor DNA may be present in a tumor sample isolated a subject.

In some embodiments, the diagnostic kit may further comprise one or more reagents for purifying tumor DNA (e.g., ctDNA) from a biological sample from a subject. In some embodiments, the one or more reagents may be used with a sequencing technique (e.g., next-generation sequencing (NGS)) to determine one or more mutations as disclosed herein.

In certain aspects is provided a diagnostic kit, wherein said diagnostic kit comprises (i) one or more reagents for determining the expression level of EGFR and/or MET in a tumor sample from a subject with a cancer, and (ii) optionally packaging and/or instructions for use. In some embodiments, the one or more reagents may be used with immunohistochemistry (IHC) to determine the expression level of EGFR and/or MET.

### EXEMPLARY EMBODIMENTS

1. A method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising
   a) determining the presence of one or more mutations in tumor DNA obtained from the subject, wherein the one or more mutations are selected from mutations in one or more genes from the RAS/RAF/MEK pathway and mutations in PIK3CA; and
   b) (i) identifying the cancer in the subject as susceptible to the treatment with the combination therapy, when tumor DNA from said subject has no said mutations, or (ii) identifying the cancer in the subject as not susceptible to the treatment with the combination therapy, when tumor DNA from said subject has one or more said mutations.
2. A method for treating a cancer in a subject in need thereof, said method comprising
   a) determining the presence of one or more mutations in tumor DNA obtained from the subject, wherein the one or more mutations are selected from mutations in one or more genes from the RAS/RAF/MEK pathway and mutations in PIK3CA; and
   b) (i) when tumor DNA from said subject has no said mutations, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI), or (ii) when tumor DNA from said subject has one or more said mutations, administering to the subject a cancer therapy which does not include the combination therapy used in (i).
3. The method of embodiment 1 or embodiment 2, wherein the one or more genes from RAS/RAF/MEK pathway are FGFR3, KRAS, BRAF, ERBB2, ALK, NRAS, PDGFRA and/or RET.
4. The method of embodiment 3, wherein the mutations in one or more genes from RAS/RAF/MEK pathway comprises FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions.
5. The method of embodiment 4, wherein the KRAS G12X mutations are KRAS G12D, KRAS G12A, KRAS G12C, and KRAS G12V.
6. The method of any one of embodiments 1-5, wherein the mutations in PIK3CA comprise PIK3CA E545K.
7. The method of any one of embodiments 1-5, wherein the one or more mutations are further selected from mutations in one or more genes from the WNT/b-catenin pathway.
8. The method of embodiment 6, wherein the one or more genes from WNT/b-catenin pathway are APC and CTNNB1.
9. The method of embodiment 7, wherein the mutations in one or more genes from WNT/b-catenin pathway comprise APC Q1469, APC R405, APC S713, CTNNB1 S33P, CTNNB1 S37C, CTNNB1 S37F, and CTNNB1 S45P.
10. A method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising
   a) determining the presence of one or more mutations in tumor DNA obtained from the subject, wherein the one or more mutations are selected from the following two groups:
      (1) PIK3CA E545K, PIK3CA E542K/V, PIK3CA H1047R, PIK3CA amplification, KRAS G12V/C/D/X (X being any amino acid other than G, V, C and D), KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletions, PTEN N48K, CDKN2A G101W, CDKN2B mutations, ALK fusions, FGFR3-TACC3 fusion, TPM3-NTRK1 fusion, RET fusions, BRAF fusions, and other oncogenic fusion events;
      (2) EGFR C797S, EGFR L792H, EGFR amplification, EGFR G796S, EGFR L718X (X being any amino acid), EGFR E709K, EGFR G724S, MET amplification, and MET exon 14 skipping (METex14) mutations; and
   b) (i) identifying the cancer in the subject as susceptible to the treatment with the combination therapy, when tumor DNA from said subject has either no mutations from group (1), or has one or more mutations from group (1) and one or more mutations from group (2), or (ii) identifying the cancer in the subject as not susceptible to the treatment with the combination therapy, when tumor DNA from said subject has one or more mutations from group (1) and no mutations from group (2).
11. A method for treating a cancer in a subject in need thereof, said method comprising
   a) determining the presence of one or more mutations in tumor DNA obtained from the subject, wherein the one or more mutations are selected from the following two groups:
      (1) PIK3CA E545K, PIK3CA E542K/V, PIK3CA H1047R, PIK3CA amplification, KRAS G12V/C/D/X, KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletion, PTEN N48K, CDKN2A G101W, CDKN2B, ALK fusions, FGFR3-TACC3 and other fusions, RET fusions, BRAF fusions, and other oncogenic fusion events;
      (2) EGFR C797S, EGFR L792H, EGFR amplification, EGFR G796S, EGFR L718X (X being any amino acid), EGFR E709K, EGFR G724S, MET amplification, and MET exon 14 skipping (METex14) mutations; and
   b) (i) when tumor DNA from said subject has either no mutations from group (1), or has one or more mutations from group (1) and one or more mutations from group (2), administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI), or (ii) when tumor DNA from said subject has one or more mutations from group (1) and no mutations from group (2), administering to the subject a cancer therapy which does not include the combination therapy used in (i).
12. The method of embodiment 10 or embodiment 11, wherein the HER2 oncogenic alterations comprise HER2 Y772 A775 duplication, HER2 L755M/S/W and HER2 S310F/Y.
13. The method of any one of embodiments 10-12, wherein the PTEN deletions comprise PTEN I33del and PTEN I14del.
14. The method of any one of embodiments 10-13, wherein the ALK fusions comprise SQSTM1-ALK fusion and EML4-ALK fusion.
15. The method of any one of embodiments 10-14, wherein the RET fusions comprise CCDC6-RET fusion, KIF5B-RET fusion, and NCOA4-RET fusion.
16. The method of any one of embodiments 1-15, wherein the cancer is a lung cancer.
17. The method of embodiment 16, wherein the lung cancer is a non-small cell lung cancer (NSCLC).
18. The method of any one of embodiments 1-17, wherein the cancer in the subject is resistant to treatment with an EGFR TKI which is not the same as the EGFR TKI used in the combination therapy.
19. The method of embodiment 18, wherein the EGFR TKI to which the cancer is resistant is selected from osimertinib, erlotinib, afatinib, rociletinib, olmutinib, and any combination thereof
20. The method of embodiment 19, wherein the EGFR TKI to which the cancer is resistant is osimertinib.
21. The method of any one of embodiments 1-20, wherein the subject is chemotherapy naïve.
22. The method of any one of embodiments 1-21, wherein the tumor DNA from the subject has at least one EGFR-activating mutation.
23. The method of embodiment 22, wherein the EGFR-activating mutation is selected from exon 19 deletions, L858R, and T790M.
24. The method of any one of embodiments 1-23, wherein the tumor DNA is circulating tumor DNA (ctDNA).
25. The method of embodiment 24, wherein the ctDNA is present in a biological sample isolated from the subject.
26. The method of embodiment 25, wherein the biological sample is a blood sample or a plasma sample.
27. The method of embodiment 25 or embodiment 26, wherein ctDNA is isolated from the biological sample prior to mutation identification.
28. The method of any one of embodiments 1-27, wherein the tumor DNA is present in a tumor sample isolated from the subject.
29. The method of embodiment 28, wherein the tumor DNA is isolated from the tumor sample prior to mutation identification.
30. The method of any one of embodiments 1-29, wherein the one or more mutations are determined by sequencing.
31. The method of embodiment 30, wherein the one or more mutations are determined using next-generation sequencing (NGS).
32. The method of any one of embodiments 1-31, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.
33. The method of embodiment 32, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.
34. The method of embodiment 32 or 33, wherein the bispecific anti-EGFR/c-Met antibody is an IgG1 isotype.
35. The method of any one of embodiments 1-34, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.
36. The method of any one of embodiment 1-35, wherein the bispecific anti-EGFR/c-Met antibody comprises a biantennary glycan structure with a fucose content of about between 1% to about 15%.
37. The method of any one of embodiments 1-36, wherein the bispecific anti-EGFR/c-Met antibody is administered intravenously to the subject.
38. The method of embodiment 37, wherein the bispecific anti-EGFR/c-Met antibody is administered at a dose of between about 140 mg to about 2240 mg.
39. The method of embodiment 38, wherein the bispecific anti-EGFR/c-Met antibody is administered at a dose of about 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1575 mg, 1600 mg, 2100 mg, or 2240 mg.
40. The method of embodiment 39, wherein the bispecific anti-EGFR/c-Met antibody is administered at a dose of 1050 mg if the subject has a body weight of less than 80 kg.
41. The method of embodiment 39, wherein the bispecific anti-EGFR/c-Met antibody is administered at a dose of 1400 mg if the subject has a body weight of greater than or equal to 80 kg.
42. The method of any one of embodiments 1-36, wherein the bispecific anti-EGFR/c-Met antibody is administered subcutaneously or intradermally to the subject.
43. The method of embodiment 42, wherein the bispecific anti-EGFR/c-Met antibody is administered subcutaneously or intradermally at a dose sufficient to achieve a therapeutic effect in the subject.
44. The method of any one of embodiments 1-43, wherein the bispecific anti-EGFR/c-Met antibody is administered twice a week, once a week, once in two weeks, once in three weeks or once in four weeks.
45. The method of any one of embodiments 1-44, wherein the EGFR TKI administered in combination with the bispecific anti-EGFR/c-Met antibody is lazertinib.
46. The method of any one of embodiments 1-45, wherein the EGFR TKI administered in combination with the bispecific anti-EGFR/c-Met antibody is administered at a dose of between about 20 to about 320 mg.
47. The method of any one of embodiments 1-46, wherein the EGFR TKI administered in combination with the bispecific anti-EGFR/c-Met antibody is administered at a dose of about 240 mg.
48. The method of any one of embodiments 1-47, wherein the EGFR TKI administered in combination with the bispecific anti-EGFR/c-Met antibody is administered daily, every other day, twice a week, or once a week.
49. The method of any one of embodiments 1-48, wherein the EGFR TKI administered in combination with the bispecific anti-EGFR/c-Met antibody is administered daily.
50. The method of any one of embodiments 1-49, wherein the EGFR TKI administered in combination with the bispecific anti-EGFR/c-Met antibody is administered orally.
51. The method of any one of embodiments 2-9 and 11-50, wherein the cancer therapy which does not include the combination therapy used in (i) is a platinum-based chemotherapy.
52. The method of embodiment 51, wherein the platinum-based chemotherapy comprises carboplatin and/or cisplatin.
53. The method of any one of embodiments 1-52, comprising obtaining a biological sample from the subject prior to step (a), wherein said biological sample comprises tumor DNA, and optionally purifying said tumor DNA from said biological sample.
54. A method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising
   a) determining expression level of EGFR or MET in a tumor sample obtained from the subject using immunohistochemistry (IHC),
   b) determining a staining intensity score, on a scale from 0 to 3+, based on the expression level of EGFR or MET determined in step (a), and
   c) (i) identifying the cancer in the subject as susceptible to the treatment with the combination therapy, when the staining intensity score is 3+, or (ii) identifying the cancer in the subject as not susceptible to the treatment with the combination therapy, when the staining intensity score is less than 3+.
55. The method of embodiment 54, wherein in step c) identifying the cancer in the subject as susceptible to the treatment with the combination therapy, when the staining intensity score is 3+ in greater than or equal to 25% of the cells of the tumor sample, or (ii) identifying the cancer in the subject as not susceptible to the treatment with the combination therapy, when the staining intensity score is 3+ in less than 25% cells of the tumor sample.
56. A method for treating a cancer in a subject in need thereof, said method comprising
   a) determining the expression level of EGFR or MET in a tumor sample obtained from the subject using immunohistochemistry (IHC),
   b) determining a staining intensity score, on a scale from 0 to 3+, based on the expression level of EGFR or MET determined in step (a), and
   c) (i) when the staining intensity score is 3+, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI); or (ii) when the staining intensity score is less than 3+, not administering to the subject the combination therapy used in (i) or administering to the subject a cancer therapy which does not include the combination therapy used in (i).
57. The method of embodiment 56, wherein in step (c), (i) when the staining intensity score is 3+ in greater than or equal to 25% of the cells of the tumor sample, administering to the subject a therapeutically effective amount of the combination therapy; or (ii) when the staining intensity score is 3+ in less than 25% cells of the tumor sample, not administering to the subject the combination therapy used in (i) or administering to the subject a cancer therapy which does not include the combination therapy used in (i).
58. A method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising
   a) determining the expression level of EGFR and MET in a tumor sample obtained from the subject using immunohistochemistry (IHC),
   b) calculating a combined H score based on the expression level of EGFR and MET determined in step (a), and
   c) (i) identifying the cancer in the subject as susceptible to the treatment with the combination therapy, when the combined H score is greater than or equal to 400, or (ii) identifying the cancer in the subject as not susceptible to the treatment with the combination therapy, when the combined H score is less than 400.
59. A method for treating a cancer in a subject in need thereof, said method comprising
   a) determining the expression level of EGFR and MET in a tumor sample obtained from the subject using immunohistochemistry (IHC),
   b) calculating a combined H score based on the expression level of EGFR and MET determined in step (a), and
   c) (i) when the combined H score is greater than or equal to 400, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI); (ii) when the combined H score is less than 400, not administering to the subject the combination therapy used in (i) or administering to the subject a cancer therapy which does not include the combination therapy used in (i).
60. The method of any one of embodiments 54-59, wherein the cancer is a lung cancer.
61. The method of embodiment 60, wherein the lung cancer is a non-small cell lung cancer (NSCLC).
62. The method of any one of embodiments 54-61, wherein the cancer in the subject is resistant to treatment with an EGFR TKI which is not the same as the EGFR TKI used in the combination therapy.
63. The method of embodiment 62, wherein the EGFR TKI to which the cancer is resistant is selected from osimertinib, erlotinib, afatinib, rociletinib, olmutinib, and any combination thereof
64. The method of embodiment 63, wherein the EGFR TKI to which the cancer is resistant is osimertinib.
65. The method of any one of embodiments 54-64, wherein the subject is chemotherapy naïve.
66. The method of any one of embodiments 54-65, wherein the tumor of the subject has at least one EGFR-activating mutation.
67. The method of embodiment 66, wherein the EGFR-activating mutation is selected from exon 19 deletions, L858R, and T790M.
68. The method of any one of embodiments 64-67, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.
69. The method of embodiment 68, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.
70. The method of embodiment 68 or 69, wherein the bispecific anti-EGFR/c-Met antibody is an IgG1 isotype.
71. The method of any one of embodiments 68-70, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.
72. The method of any one of embodiment 54-71, wherein the bispecific anti-EGFR/c-Met antibody comprises a biantennary glycan structure with a fucose content of about between 1% to about 15%.
73. The method of any one of embodiments 54-72, wherein the bispecific anti-EGFR/c-Met antibody is administered intravenously to the subject.
74. The method of embodiment 73, wherein the bispecific anti-EGFR/c-Met antibody is administered at a dose of between about 140 mg to about 2240 mg.
75. The method of embodiment 74, wherein the bispecific anti-EGFR/c-Met antibody is administered at a dose of about 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1575 mg, 1600 mg, 2100 mg, or 2240 mg.
76. The method of embodiment 75, wherein the bispecific anti-EGFR/c-Met antibody is administered at a dose of 1050 mg if the subject has a body weight of less than 80 kg.
77. The method of embodiment 75, wherein the bispecific anti-EGFR/c-Met antibody is administered at a dose of 1400 mg if the subject has a body weight of greater than or equal to 80 kg.
78. The method of any one of embodiments 54-72, wherein the bispecific anti-EGFR/c-Met antibody is administered subcutaneously or intradermally to the subject.
79. The method of embodiment 78, wherein the bispecific anti-EGFR/c-Met antibody is administered subcutaneously or intradermally at a dose sufficient to achieve a therapeutic effect in the subject.
80. The method of any one of embodiments 54-79, wherein the bispecific anti-EGFR/c-Met antibody is administered twice a week, once a week, once in two weeks, once in three weeks or once in four weeks.
81. The method of any one of embodiments 54-80, wherein the EGFR TKI administered in combination with the bispecific anti-EGFR/c-Met antibody is lazertinib.
82. The method of any one of embodiments 54-81, wherein the EGFR TKI administered in combination with the bispecific anti-EGFR/c-Met antibody is administered at a dose of between about 20 to about 320 mg.
83. The method of any one of embodiments 54-82, wherein the EGFR TKI administered in combination with the bispecific anti-EGFR/c-Met antibody is administered at a dose of about 240 mg.
84. The method of any one of embodiments 54-83, wherein the EGFR TKI administered in combination with the bispecific anti-EGFR/c-Met antibody is administered daily, every other day, twice a week, or once a week.
85. The method of any one of embodiments 54-84, wherein the EGFR TKI administered in combination with the bispecific anti-EGFR/c-Met antibody is administered daily.
86. The method of any one of embodiments 54-85, wherein the EGFR TKI administered in combination with the bispecific anti-EGFR/c-Met antibody is administered orally.
87. The method of any one of embodiments 56-57 and 59-86, wherein the cancer therapy which does not include the combination therapy used in (i) is a platinum-based chemotherapy.
88. The method of embodiment 87, wherein the platinum-based chemotherapy comprises carboplatin and/or cisplatin.
89. The method of any one of embodiments 54-88, comprising obtaining a tumor sample from the subject prior to step (a).
90. A diagnostic kit comprising (i) one or more reagents for determining the presence of one or more mutations in tumor DNA from a subject with a cancer, and (ii) optionally packaging and/or instructions for use, wherein the one or more mutations are selected from mutations in one or more genes from the RAS/RAF/MEK pathway and mutations in PIK3CA.
91. The diagnostic kit of embodiment 90, wherein the one or more genes from RAS/RAF/MEK pathway are FGFR3, KRAS, BRAF, ERBB2, ALK, NRAS, PDGFRA and/or RET.
92. The diagnostic kit of embodiment 91, wherein the mutations in one or more genes from RAS/RAF/MEK pathway comprise FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions.
93. The diagnostic kit of embodiment 92, wherein the KRAS G12X mutations are KRAS G12D, KRAS G12A, KRAS G12C, and KRAS G12V.
94. The diagnostic kit of any one of embodiments 90-93, wherein the mutations in PIK3CA comprise PIK3CA E545K.
95. The diagnostic kit of any one of embodiments 90-94, wherein the one or more mutations are further selected from mutations in one or more genes from the WNT/b-catenin pathway.
96. The diagnostic kit of embodiment 95, wherein the one or more genes from WNT/b-catenin pathway are APC and CTNNB1.
97. The diagnostic kit of embodiment 96, wherein the mutations in one or more genes from WNT/b-catenin pathway comprise APC Q1469, APC R405, APC 5713, CTNNB1 S33P, CTNNB1 S37C, CTNNB1 S37F, and CTNNB1 S45P.
98. A diagnostic kit comprising (i) one or more reagents for determining the presence of one or more mutations in tumor DNA from a subject with a cancer, and (ii) optionally packaging and/or instructions for use, wherein the one or more mutations are selected from the following two groups:
   (1) PIK3CA E545K, PIK3CA E542K/V, PIK3CA H1047R, PIK3CA amplification, KRAS G12V/C/D/X (X being any amino acid other than G, V, C and D), KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletions, PTEN N48K, CDKN2A G101W, CDKN2B mutations, ALK fusions, FGFR3-TACC3 fusion, TPM3-NTRK1 fusion, RET fusions, BRAF fusions, and other oncogenic fusion events; and
   (2) EGFR C797S, EGFR L792H, EGFR amplification, EGFR G796S, EGFR L718X (X being any amino acid), EGFR E709K, EGFR G724S, MET amplification, and MET exon 14 skipping (METex14) mutations.
99. The method of embodiment 98, wherein the HER2 oncogenic alterations comprise HER2 Y772 A775 duplication, HER2 L755M/S/W and HER2 S310F/Y.
100. The method of embodiments 98 or 99, wherein the PTEN deletions comprise PTEN 133del and PTEN I14del.
101. The method of any one of embodiments 98-99, wherein the ALK fusions comprise SQSTM1-ALK fusion and EML4-ALK fusion.
102. The method of any one of embodiments 98-101, wherein the RET fusions comprise CCDC6-RET fusion, KIF5B-RET fusion, and NCOA4-RET fusion.
103. The diagnostic kit of any one of embodiments 90-102, wherein the tumor DNA is circulating tumor DNA (ctDNA).
104. The diagnostic kit of embodiment 103, wherein the ctDNA is present in a biological sample isolated from the subject.
105. The diagnostic kit of embodiment 104, wherein the biological sample is a blood sample or a plasma sample.
106. The diagnostic kit of any one of embodiments 90-102, wherein the tumor DNA is present in a tumor sample isolated from the subject.
107. The diagnostic kit of any one of embodiments 103-106, further comprising one or more reagents for purifying said tumor DNA from said biological sample from the subject.
108. The diagnostic kit of any one of embodiments 90-107, wherein the one or more reagents can be used with a sequencing technique to determine the one or more mutations.
109. The diagnostic kit of any one of embodiments 90-108, wherein the one or more reagents can be used with next-generation sequencing (NGS) to determine the one or more mutations.
110. A diagnostic kit comprising (i) one or more reagents for determining the expression level of EGFR and/or MET in a tumor sample from a subject with a cancer, and (ii) optionally packaging and/or instructions for use.
111. The diagnostic kit of embodiment 110, wherein the one or more reagents can be used with immunohistochemistry (IHC) to determine the expression level of EGFR and/or MET.

### EXAMPLES

The following examples are provided to further describe some of the embodiments disclosed herein. The examples are intended to illustrate, not to limit, the disclosed embodiments.

### Example 1. Amivantamab in Combination with Lazertinib for the Treatment of Osimertinib-Relapsed, Chemotherapy-Naïve EGFR Mutant (EGFRm) Non-Small Cell Lung Cancer (NSCLC) and Potential Biomarkers for Response

The present Example investigated the preliminary efficacy of the combination of amivantamab, an epidermal growth factor receptor (EGFR) and mesenchymal-epithelial transition factor (MET) bispecific antibody, and lazertinib, a third-generation tyrosine kinase inhibitor (TKI), in treatment-naïve and osimertinib (osi)-relapsed patients with EGFR mutant (EGFRm) non-small cell lung cancer (NSCLC). Briefly, pre-treatment tumor biopsies and circulating tumor DNA (ctDNA) were collected prior to administration of amivantamab in combination lazertinib. Osimertinib-resistance mutations or amplifications in EGFR/MET identified by next-generation sequencing (NGS) in either ctDNA or tumor biopsy (biomarker-positive [pos]), were evaluated for enriching response. Immunohistochemistry (IHC) staining for EGFR and MET expression was also explored as a potential biomarker for response.

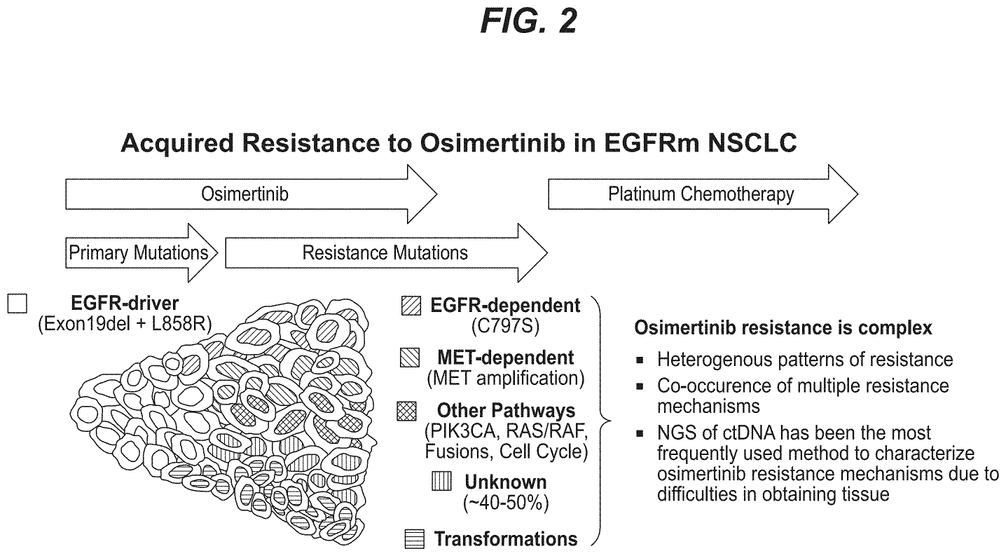

A schematic representation of the structure of amivantamab and lazertinib, and detailed description of the mechanism of action (MOA) for amivantamab is displayed in FIG. 1. Progression of acquired resistance to osimertinib in epidermal growth factor receptor mutant (EGFRm) non-small cell lung cancer (NSCLC) is exemplified in FIG. 2. Specifically, primary mutations, e.g., EGFR-driver mutations (exon 19 deletion+L858R), may co-occur together with resistance mutations, such as those that are EGFR-dependent (C797S) or MET-dependent (MET amplification); implicate other pathways (e.g., PIK3CA, RAS/RAF/MEK, Fusions, Cycle); may be attributed to transformations; or are as yet unknown (˜40-50%), each contributing to osimertinib resistance. Ultimately, the complexity of osimertinib resistance likely arises from heterogenous patterns of resistance together with co-occurrence of multiple resistance mechanisms. Sequencing of a single tumor lesion may not reveal heterogenous patterns or co-occurring mutations at resistance and, in this sense, plasma next-generation sequencing (NGS) may be more useful. NGS of circulating tumor DNA (ctDNA) has been the most frequently used method to characterize osimertinib resistance mechanisms due to difficulties in obtaining tissue (Papadimitrakopoulou et al., Annals of Oncol 29:VIII741, 2018; Ramalingam et al., Annals of Oncol 29:VII1740, 2018).

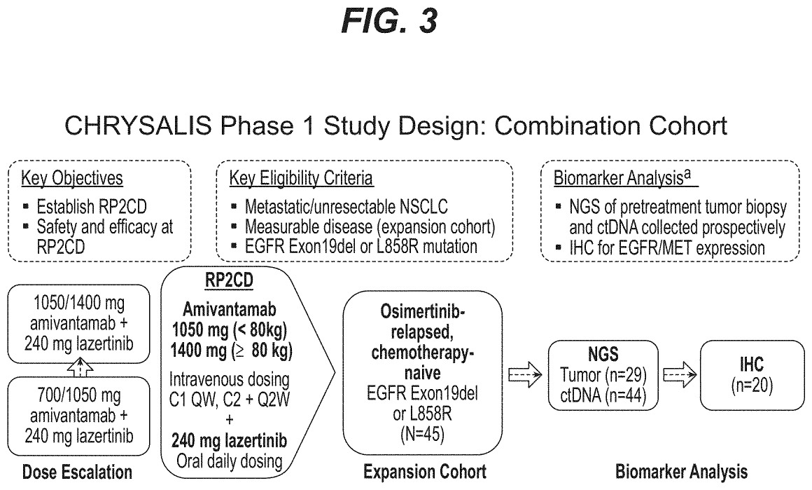

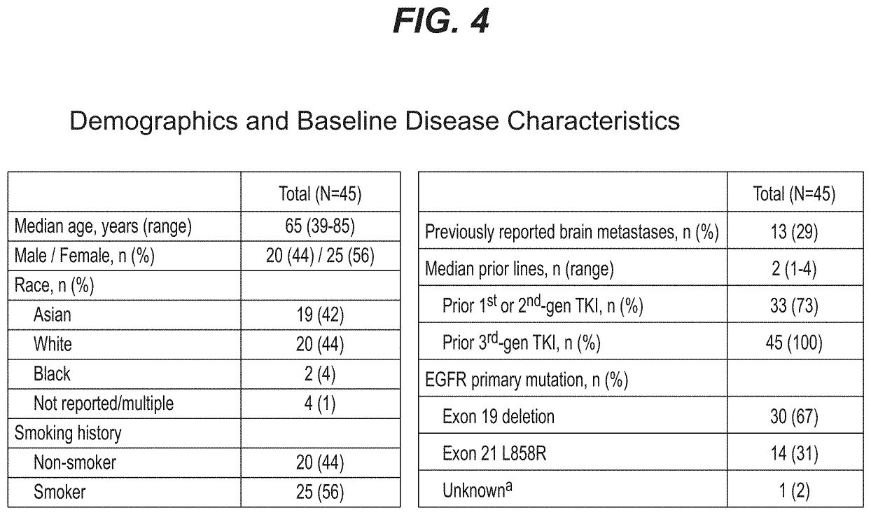

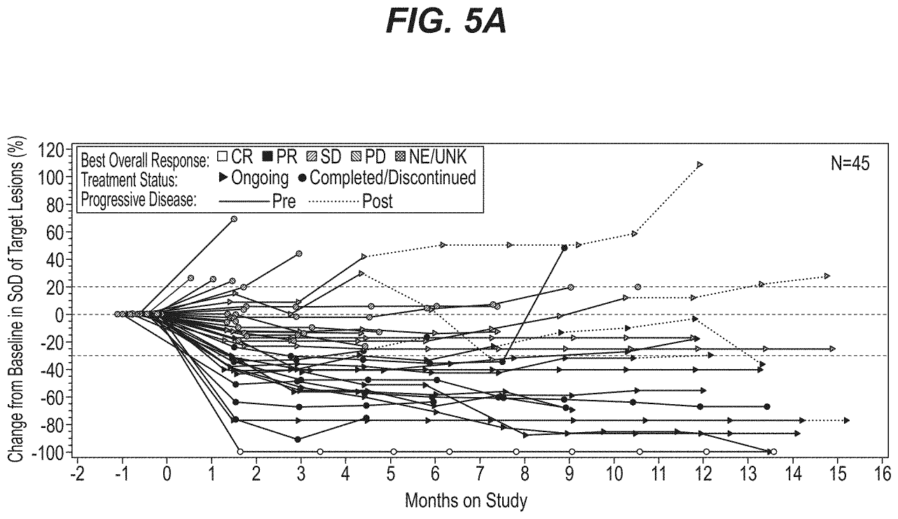

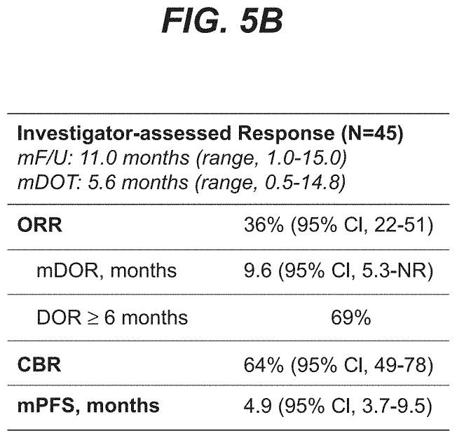

According to methods of the present disclosure, patients (N=45) with EGFR exon 19 deletion or L858R mutation NSCLC who had progressed on osimertinib without intervening chemotherapy, were enrolled in the combination cohort of the ongoing CHRYSALIS study (NCT02609776, Cohort E), as depicted in FIG. 3. A description of the patient demographics and baseline disease characteristics is shown in FIG. 4. With pre-treatment tumor biopsies and ctDNA collected prospectively, patients received the combination dose of 1050/1400 mg amivantamab+240 mg lazertinib to assess safety and efficacy in the osimertinib-relapsed population. Response was assessed by investigator per RECIST v1.1. Osimertinib-resistance mutations or amplifications in EGFR/MET identified by next-generation sequencing (NGS) in either ctDNA or tumor biopsy (biomarker-positive [pos]), were evaluated for enriching response. Durable responses were observed with combined amivantamab plus lazertinib with manageable safety (FIGS. 5A-5B). Sum of target lesion diameters (SoD) shown in FIG. 5A was measured as described in E. A. Eisenhauer et al., New response evaluation criteria in solid tumours: Revised RECIST guideline (version 1.1); European J of Cancer 45 (2009) 228-247. The safety profile was consistent with previous experience with amivantamab plus lazertinib by the inventors (Cho et al., Ann Oncol 31:S813, 2020). The most common adverse events (AE) were infusion-related reaction (IRR; 78%), rash (acneiform dermatitis, 51%+rash, 27%), and paronychia (49%), the majority of which were grade 1-2. Of treatment-related instances, 16% were grade ≥3 AE, 4% were discontinuations, and 18% were dose reductions.

Among key findings of the present Example, of the 45 osimertinib-relapsed patients, 36% (95% confidence interval [CI], 22-51) had a confirmed response (1 complete response and 15 partial responses [PR]). At a median follow-up of 8.2 months (1.0-11.8), 20/45 patients (44%) remained on treatment. With 11/16 patients (69%) continuing in response (2.6-9.6+ months), median duration of response has not been reached (NR). The median progression-free survival (mPFS) was 4.9 months (95% CI, 3.7-8.3).

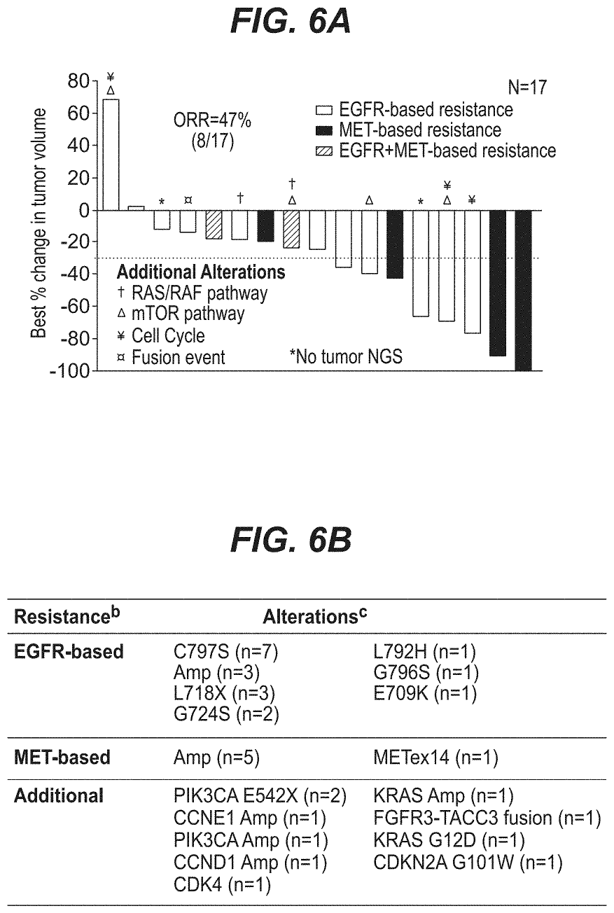

In total, 44/45 patients were evaluable by ctDNA and 29/45 by tumor NGS. Genetic testing identified 17 biomarker-positive patients, of whom 8 (47%) responded (FIGS. 6A-6B). At a median follow-up of 8.2 months (1.0-11.8), 20/45 patients (44%) remain on treatment. With 11/16 patients (69%) continuing in response (2.6-9.6+ months), median duration of response has not been reached (NR). The median progression-free survival (mPFS) was 4.9 months (95% CI, 3.7-8.3). FIG. 6A displays a plot of the best percentage change in tumor volume for EGFR-based resistance, MET-based-resistance, and EGFR plus MET (EGFR+MET)-based resistance groups. FIG. 6B shows a summary chart of the genetic alterations determined for the EGFR-based, MET-based, and additional resistance groups.

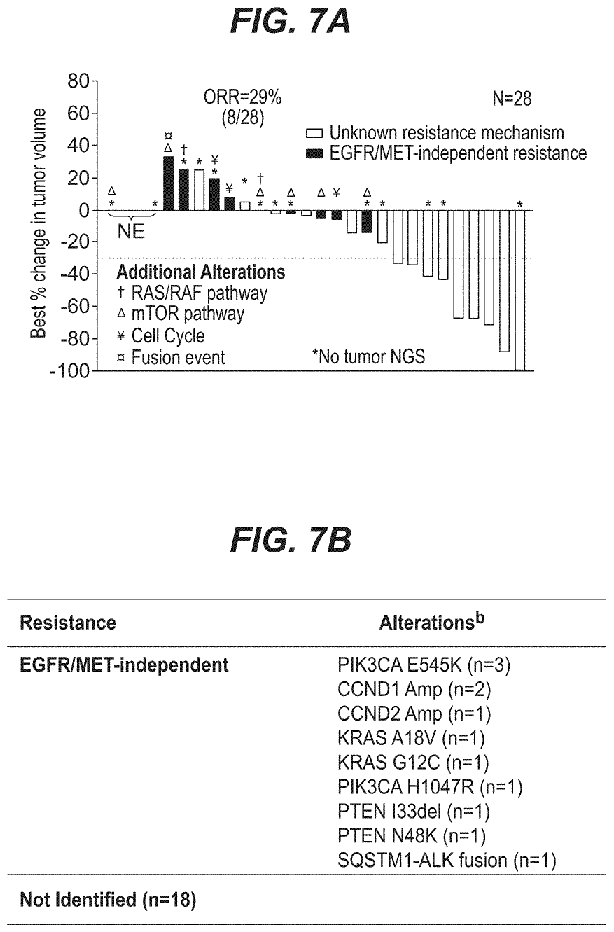

Of the remaining 28 patients, 8 (29%) responded (FIGS. 7A-7B). Among these 28 patients, 18 had unknown mechanisms of osimertinib-resistance (8 PR) and 10 had non-EGFR/MET mechanisms of resistance identified (none responded). The mPFS (95% CI) for biomarker-positive and remaining patients was 6.7 months (3.4-NR) and 4.1 months (1.4-9.5), respectively. FIG. 7A displays a plot of the best percentage change in tumor volume for unknown resistance mechanism and EGFR/MET-independent resistance groups. FIG. 7B shows a summary chart of a subset of genetic alterations, e.g., mutations, determined for the EGFR/MET-independent group. As a non-limiting example, exemplary EGFR/MET-independent genetic alterations may include PIK3CA E545K, PIK3CA E542K/V, PIK3CA H1047R, PIK3CA amplification, KRAS G12V/C/D/X, KRAS amplification, BRAF V600E, BRAF amplification, CCND1 amplification, CCND2 amplification, CCNE1 amplification, CDK4 amplification, CDK6 amplification, HER2 amplification, HER2 oncogenic alterations, PTEN deletion, PTEN N48K, CDKN2A G101W, CDKN2B, ALK fusions, FGFR3-TACC3 and other fusions, RET fusions, BRAF fusions, and other oncogenic fusion events. According to the present patient stratification method, in the absence of such EGFR/MET-independent mutations, a patient may be a candidate for amivantamab and lazertinib combination treatments disclosed herein. Patients determined to have a non-EGFR non-MET (i.e., EGFR/MET-independent) mechanism of resistance, as exemplified in FIGS. 7A-7B, and additionally lack EGFR-based resistance mutations, e.g., EGFR C797S, EGFR L792H, EGFR amplification, EGFR G796S, EGFR L718X (X being any amino acid), EGFR E709K, and EGFR G724S, and/or MET-based resistance mutations, e.g., MET amplification, and MET exon 14 skipping (METex14) mutations (e.g., see FIG. 6B), would be excluded from treatment with amivantamab in combination with lazertinib treatments. These data indicate that such patients are at a low probability of responding to this combination, and given the current standard of care, they would instead be treated with e.g., platinum-based chemotherapy.

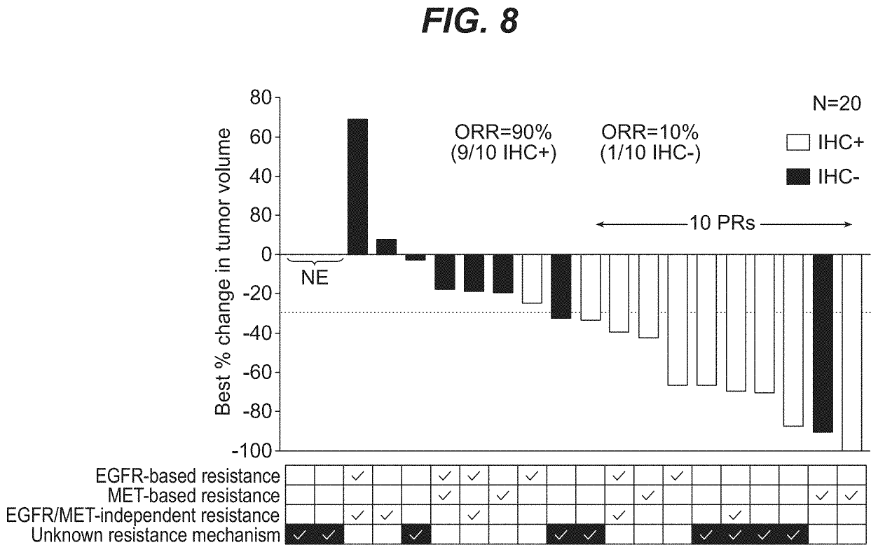

Adequate tissue was available for 20 patients to perform IHC testing for EGFR and MET (FIG. 8). Ten patient biopsy samples were immune-positive (IHC+) for EGFR/MET, exhibiting a combined EGFR plus MET (EGFR+MET) H score ≥400. The remaining ten patient biopsy samples were defined as IHC−. IHC+ patients had an overall response rate (ORR) of 90% (9/10 patients); median duration of response (mDOR) of 9.7 months; clinical benefit rate (CBR) of 100%; and, median progression-free survival (mPFS) of 12.5 months. Five responders in the IHC+ group had an unknown genetic mechanism. A high representation of positive responders (PRs) was observed in the IHC+ group, and was associated with greater percentage reduction in tumor volume. These data suggest that IHC of MET and/or EGFR, indicative of high expression, is a positive predictor of treatment response to combined amivantamab/lazertinib treatment.

The present Example demonstrated treatment with the combination of amivantamab and lazertinib yielded responses in 36% of chemotherapy-naïve patients who progressed on osimertinib. Among these patients, genetic EGFR and MET-based biomarkers of resistance identified a subgroup of patients more likely to respond to amivantamab and lazertinib, although additional patients lacking identified resistance markers also responded. An IHC-based approach may identify patients most likely to benefit from the combination regimen.

### Example 2. Validation of Biomarkers for Response in Expansion Cohort Study

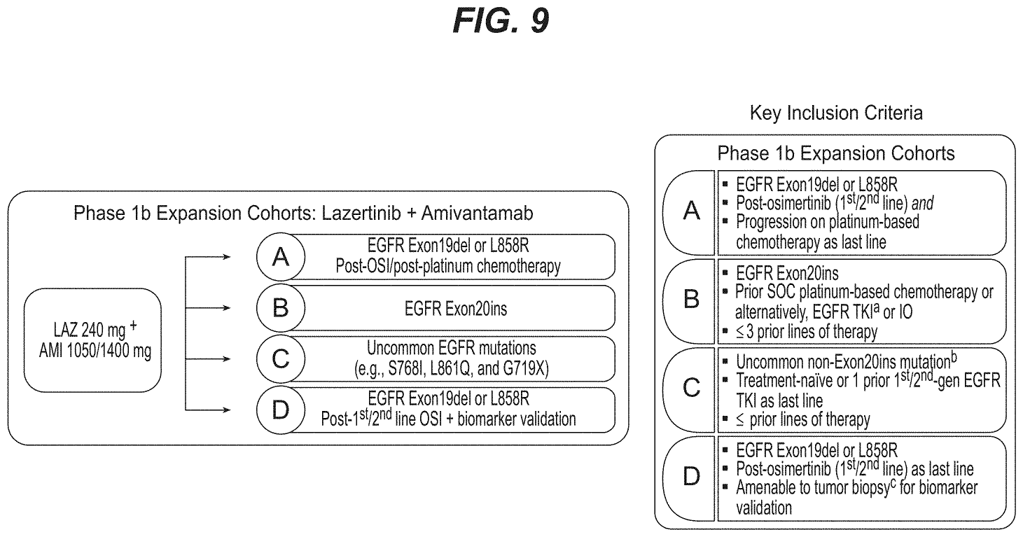

CHRYSALIS-2 phase 1/1b expansion cohort is generally performed according to an exemplary study design, as shown in FIG. 9. Key patient inclusion criteria of the phase 1b expansion cohorts A-D are applied as follows: The inclusion criteria for expansion cohort A are EGFR exon 19 deletion or L858R, post-osimertinib (1^{st}/2^{nd} line) and, progression on platinum-based chemotherapy as last line. The inclusion criterion for expansion cohort B are EGFR exon 20 insertion, prior standard of care (SOC) platinum-based chemotherapy or alternatively, EGFR TKI, which may include investigational EGFR-TKI targeting the exon 20 insertion (e.g., mobocertinib and poziotinib) or immuno-oncology therapy (TO) and, ≤3 prior lines of therapy chemotherapy as last line. The inclusion criterion for expansion cohort C are uncommon non-Exon 20 insertion mutations (e.g., S7681, L861Q, G719X), treatment-naïve or one prior 1^{st}/2^{nd}-generation EGFR TKI as last line, and ≤2 prior lines of therapy. The inclusion criteria for expansion cohort D are EGFR Exon 19 deletion or L858R, post-osimertinib (1^{st}/2^{nd} line) as last line, and amenable to tumor biopsy, following progression on the most recent system treatment or from initial biopsy in metastatic setting, for biomarker validation. Phase 1b expansion cohorts are administered lazertinib (240 mg) in combination with amivantamab 1050/1400 mg (1050 mg, body weight of <80 kg; 1400 mg, body weight of ≥80 kg). For expansion cohort D, osimertinib-resistance mutations or amplifications in EGFR/MET as exemplified in Example 1 are validated by next-generation sequencing (NGS) in either ctDNA or tumor biopsy (biomarker-positive [pos]), and evaluated for enriching response. Immunohistochemistry (IHC) staining for EGFR and MET expression are additionally validated as a biomarker for response.

### Example 3. Biomarker Strategy Based on NGS Analysis of Baseline Plasma ctDNA

### Materials and Methods

To assess a biomarker strategy that identifies patients at increased, or decreased, probability of tumor response to the treatment with amivantamab and lazertinib combination in participants previously tested positive for EGFR Exon19del or L858R mutated NSCLC, who progressed on or after osimertinib (Phase 1b expansion Cohort D), ORR per RECIST v1.1 was assessed in populations dichotomized by next generation sequencing (NGS) analysis of baseline plasma circulating tumor DNA (ctDNA). The RECIST v1.1 guideline is described in E. A. Eisenhauer et al., New response evaluation criteria in solid tumours: Revised RECIST guideline (version 1.1); European J of Cancer 45 (2009) 228-247, which is incorporated herein by reference in its entirety. These participants were previously tested positive for either EGFR Exon19del or L858R mutated NSCLC; they progressed on or after osimertinib, and were enrolled without biomarker selection and were required to submit plasma for ctDNA NGS analysis. Each patient received a classification based on the mechanisms of osimertinib resistance determined by ctDNA NGS analysis. A patient was classified as NGS1 if ctDNA NGS analysis identified the pathogenic PIK3CA E545K mutation or pathogenic alterations in the RAS/RAF/MEK pathway. A patient was classified as NGS2 if ctDNA NGS analysis identified the pathogenic PIK3CA E545K mutation or pathogenic alterations in the RAS/RAF/MEK pathway or pathogenic alterations in the WNT/b-catenin pathway. A patient was classified as NGS3 if ctDNA NGS analysis identified the pathogenic PIK3CA E545K mutation or pathogenic alterations in the RAS/RAF/MEK pathway or pathogenic alterations in the WNT/b-catenin pathway or if ctDNA NGS failed to detect the EGFR L858R mutation or an EGFR Exon 19 deletion mutation (likely due to sensitivity limitations of ctDNA assay). Progression free survival (PFS) was also compared between each NGS group.

A complete list of mutations observed in patients treated with amivantamab and lazertinib is provided below.

TABLE 1

| | | Number of |
|---|---|---|
| Mutation | Pathway | patients |
| EGFR_L858R | EGFR_DRIVER | 34 |
| EGFR_E746A_750DEL | EGFR_DRIVER | 31 |
| EGFR_L747P7_53DELINSS* | EGFR_DRIVER | 8 |
| PIK3CA_E545K | NA | 7 |
| EGFR_E746_S752DELINSV* | EGFR_DRIVER | 4 |
| EGFR_L747_A750DELINSP | EGFR_DRIVER | 4 |
| FGFR3_FUSION | RAS_RAF MEK | 3 |
| KRAS_G12D | RAS_RAF_MEK | 3 |
| BRAF_G469A | RAS_RAF_MEK | 2 |
| BRAF_V600E | RAS_RAF_MEK | 2 |
| EGFRL_747_T751DEL | EGFR_DRIVER | 2 |
| ERBB2_COPYNUMBER | RAS_RAF_MEK | 2 |
| ALK_FUSION | RAS_RAF_MEK | 1 |
| APC_Q1469. | WNT_B_CATENIN | 1 |
| APC_R405. | WNT_B_CATENIN | 1 |
| APC_S713. | WNT_B_CATENIN | 1 |
| CTNNB1_S33P | WNT_B_CATENIN | 1 |
| CTNNB1_S37C | WNT_B_CATENIN | 1 |
| CTNNB1_S37F | WNT_B_CATENIN | 1 |
| CTNNB1_S45P | WNT_B_CATENIN | 1 |
| EGFR_E746_P753DELINSVS* | EGFR_DRIVER | 1 |
| EGFR_E746_T751DELINSA* | EGFR_DRIVER | 1 |
| EGFR_E746_T751DELINSL* | EGFR_DRIVER | 1 |
| EGFR_L747_E749DEL | EGFR_DRIVER | 1 |
| EGFR_L747_K754DELINSATSPE* | EGFR_DRIVER | 1 |
| EGFR_L747_K754DELINSSN* | EGFR_DRIVER | 1 |
| EGFR_L747_S752DEL | EGFR_DRIVER | 1 |
| EGFR_L747_T751DELINSP* | EGFR_DRIVER | 1 |
| EGFR_T751_I759DELINSN* | EGFR_DRIVER | 1 |
| ERBB2_I767M | RAS_RAF_MEK | 1 |
| ERBB2_V777L | RAS_RAF_MEK | 1 |
| KRAS_A18V | RAS_RAF_MEK | 1 |
| KRAS_COPYNUMBER | RAS_RAF_MEK | 1 |
| KRAS_G12A | RAS_RAF_MEK | 1 |
| KRAS_G12C | RAS_RAF_MEK | 1 |
| KRAS_G12V | RAS_RAF_MEK | 1 |
| NRAS_Q61R | RAS_RAF_MEK | 1 |
| PDGFRA_COPYNUMBER | RAS_RAF_MEK | 1 |
| RET_FUSION | RAS_RAF_MEK | 1 |

*The mutations ending with “DELINSX” mean that the amino acids at the described positions, inclusive, were deleted and replaced with an insertion of residue(s) “X”. For example, “EGFRL747 P753DELINSS” means that the amino acids at positions 747-753, inclusive, were deleted and replaced with an insertion of “S”.

### Results

TABLE 2

| | | |
|---|---|---|
| | | NGS3 |
| | | altered |
| | | RAS/RAF/MEK, or |
| | | PIK3CA_E545K, or |
| | NGS2 | altered WNT/β-Catenin, |
| NGS1 | altered | or EGFR L858R not |
| altered | RAS/RAF/MEK, or | detected, or EGFR |
| RAS/RAF/MEK, or | PIK3CA_E545K, or | Exon 19 deletion not |
| PIK3CA_E545K | altered WNT/β-Catenin | detected |

| | Positive | Negative | Positive | Negative | Positive | Negative |
|---|---|---|---|---|---|---|
| | (23%) | (77%) | (30%) | (70%) | (40%) | (60%) |
| Response | 1 | 30 | 2 | 29 | 4 | 27 |
| Non- | 22 | 46 | 28 | 40 | 36 | 32 |
| response | | | | | | |
| Total | 23 | 76 | 30 | 69 | 40 | 59 |
| ORR | 4.3% | 39.5% | 6.7% | 42% | 10.0% | 45.8% |

Exclusion of NGS1, NGS2 or NGS3 positive patients enriched for PR and uPR relative to the unselected population. When treating a uPR as a PR, the ORR for the ORR for the NGS1, NGS2 and NGS3 negative populations are 39.5% (95% C.I.: 28.4%-51.4%), 42% (95% C.I.: 28.4%-51.4%) and 45.8% (95% C.I.: 32.7%-59.3%), respectively (see Table 2). While the ORR for the NGS1, NGS2 and NGS3 positive populations are 4.3% (95% C.I.: 0.1%-22.0%), 6.7% (95% C.I.: 0.8%-22.1%), and 10% (95% C.I.: 2.8%-23.7%), respectively (Table 2). The ORR for the unselected population of subjects with evaluable ctDNA NGS results is 31.3% (95% C.I.: 22.4%-41.4%) (Table 3).

TABLE 3
Observed ORR by cohort and by pre-determined osimertinib resistance
features

| | Cohort E* (%) | Cohort D (%) | Cohort E + D (%) |
|---|---|---|---|
| Resistance | N = 41 | N = 58 | N = 99 |
| Dependent ^{#} | 44.4 | 40.0 | 42.9 |
| Unknown ^{Δ} | 47.1 | 31.3 | 36.7 |
| Depend & Independ | 40.0 | 16.7 | 27.3 |
| Independent \hat{ } | 0.0 | 26.7 | 16.0 |
| All Subjects | 34.1 | 29.3 | 31.3 |

*Cohort E was describee in Example 1.
^{#} Dependent = EGFR/ME T dependent, i.e., EGFR C797S mutation or MET amplification.
\hat{ } Independent = EGFR/N ET independent, e.g., KRAS or PIK3CA etc.
^{Δ} Unknown = does not fi Dependent or Independent categories.

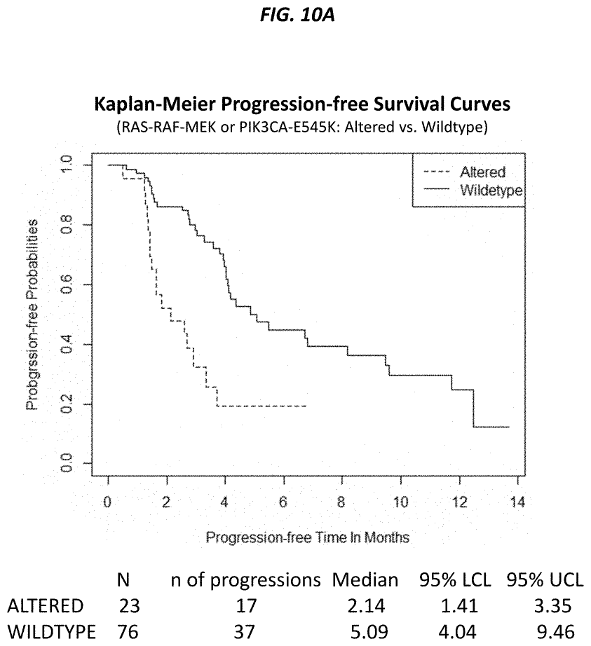

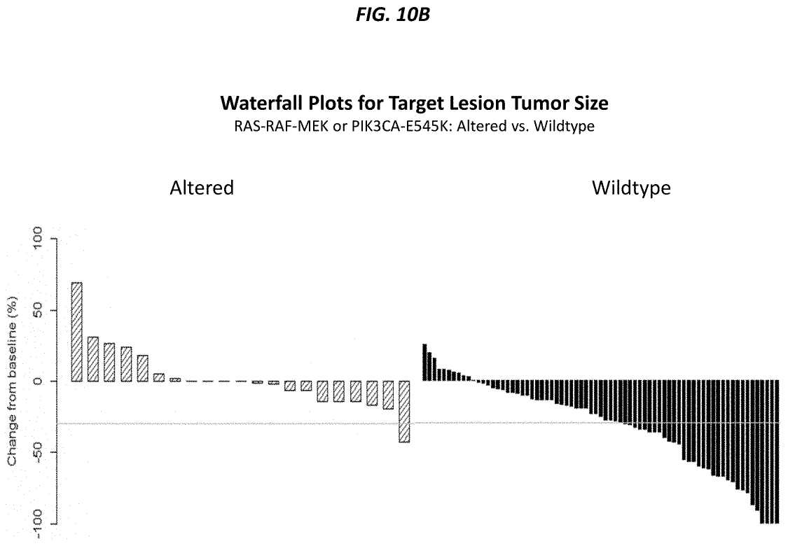

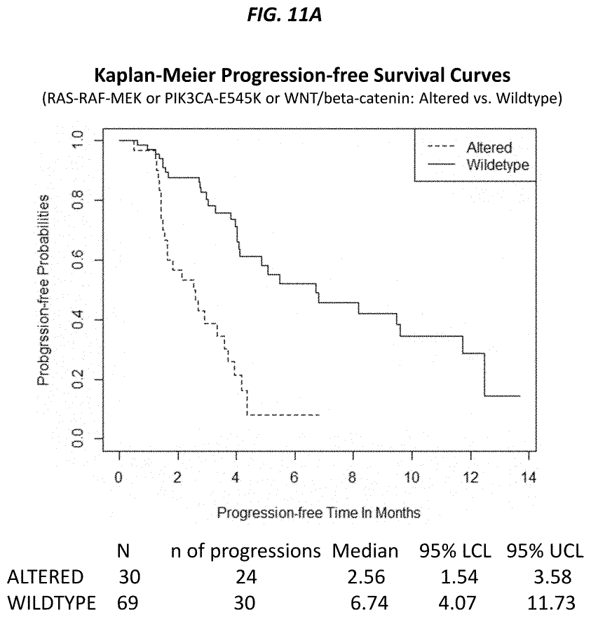

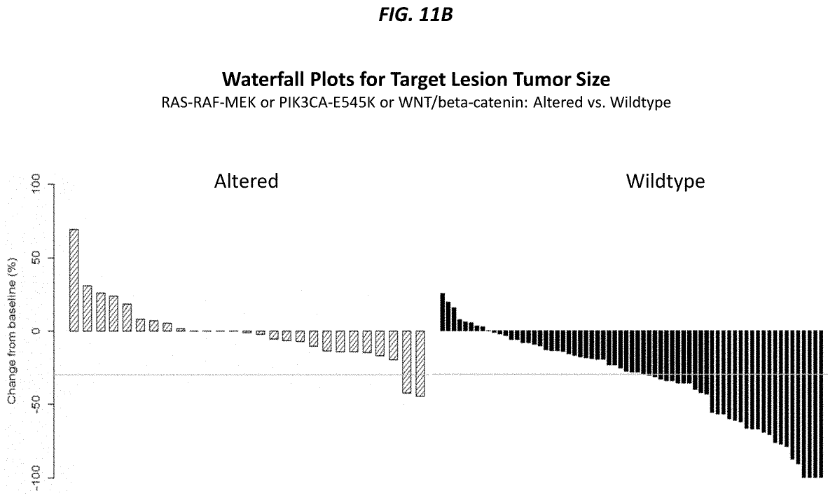

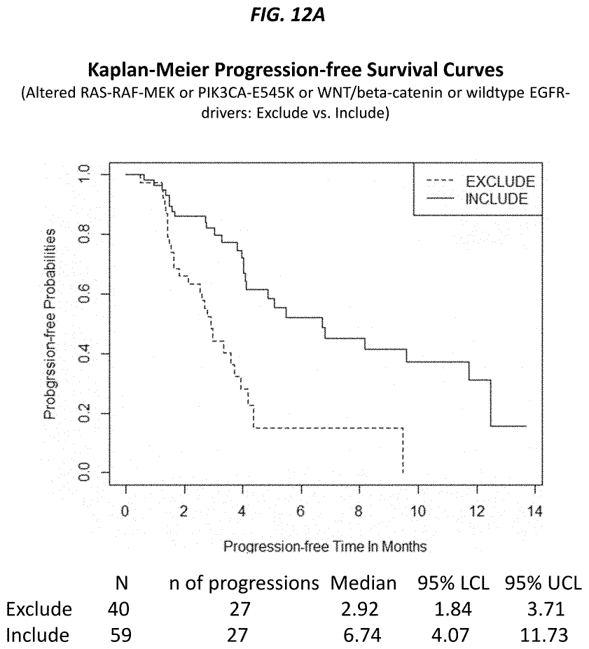

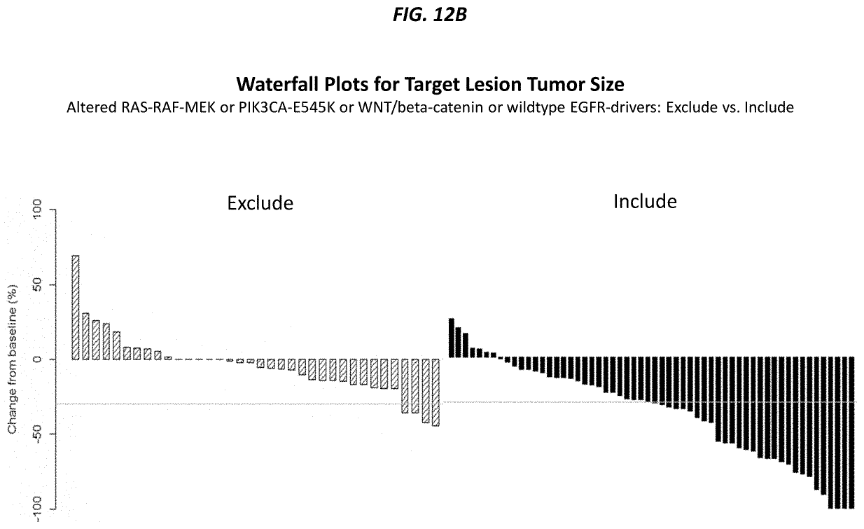

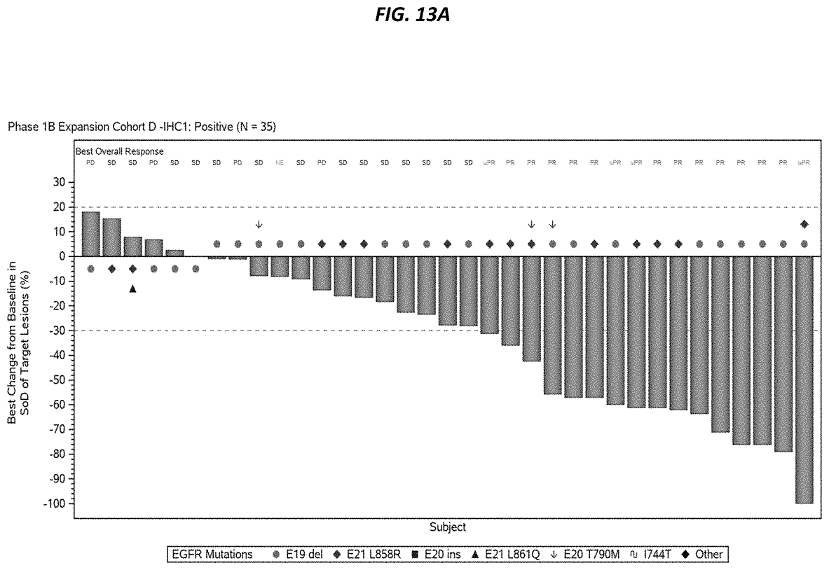

In support of the ORR, waterfall plots demonstrate an enrichment in best change from baseline of target lesion size by at least a 30% decrease in all NGS negative groups (FIGS. 10B, 11B, and 12B). At 3 months, all NGS negative groups show better PFS survival relative to their respective positive groups (FIGS. 10A, 11A, and 12A).

### Example 4. Biomarker Strategy Based on IHC Analysis of Baseline Tumor Biopsy

### Materials and Methods

To assess a biomarker strategy that identifies patients at increased, or decreased, probability of tumor response when treated with amivantamab and lazertinib combination in participants previously tested positive for EGFR Exon19del or L858R mutated NSCLC, who progressed on or after osimertinib (Phase 1b expansion Cohort D), ORR per RECIST v1.1 was assessed in populations dichotomized by Immunohistochemical (IHC) analysis of baseline tumor biopsy EGFR and MET expression. These participants previously tested positive for EGFR Exon19del or L858R mutated NSCLC, and progressed on or after osimertinib; these patients were enrolled without biomarker selection and were required to submit tumor tissue for IHC analysis (EGFR and MET expression). Each patient received a classification for each IHC assay. A patient was classified as EGFR positive (IHC1) if had an EGFR 3+ IHC intensity score per cell of 25% of the cells or greater, likewise a patient was classified as MET positive (IHC2) if their biopsy sample had a MET 3+ IHC intensity score of 25% or greater. Progression free survival was also compared between each IHC group.

### Results

TABLE 4
Response per RECIST v1.1 dichotomized by each IHC

| | IHC1 | IHC1 | IHC2 | IHC2 |
|---|---|---|---|---|
| | NEGATIVE | POSITIVE | NEGATIVE | POSITVE |
| Not | 1 | 1 | 2 | 0 |
| Evaluable/Unknown | | | | |
| Partial Response | 0 | 12 | 3 | 9 |
| (PR) | | | | |
| Progressive Disease | 3 | 4 | 5 | 2 |
| (PD) | | | | |
| Stable Disease (SD) | 11 | 14 | 19 | 6 |
| Unconfirmed Partial | 0 | 4 | 3 | 1 |
| Response (uPR) | | | | |

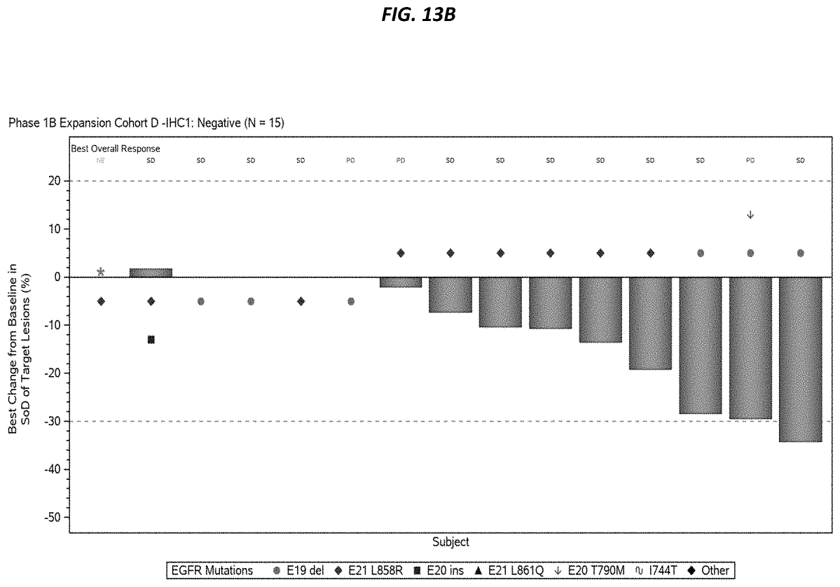

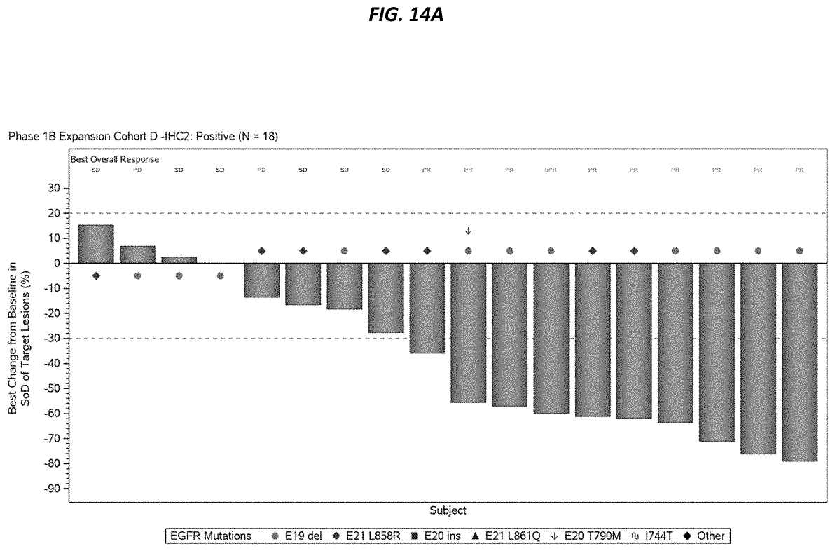

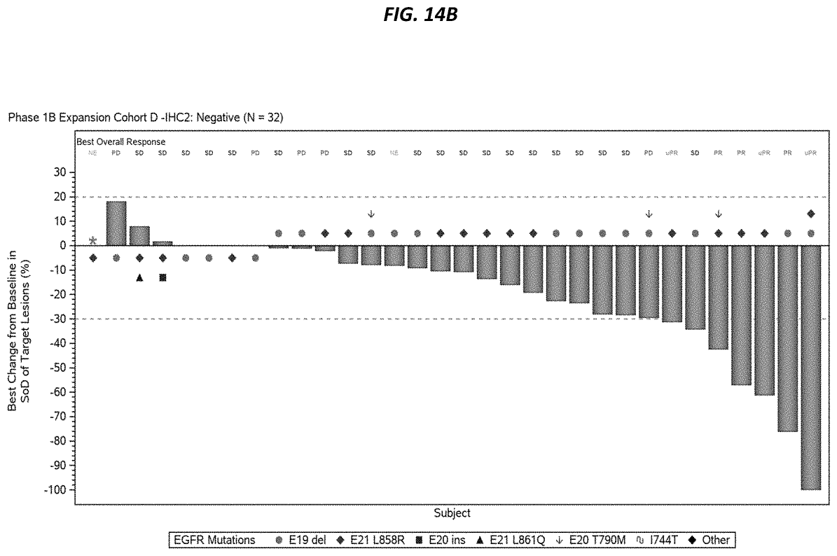

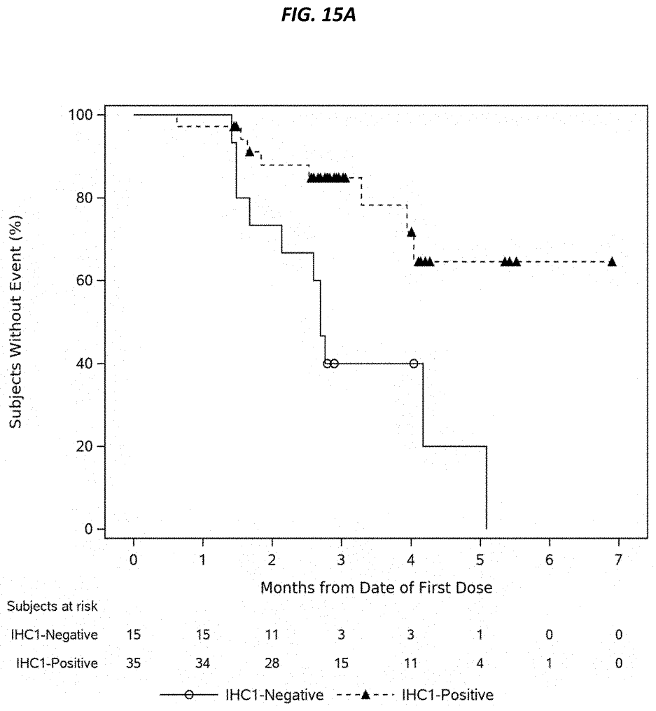

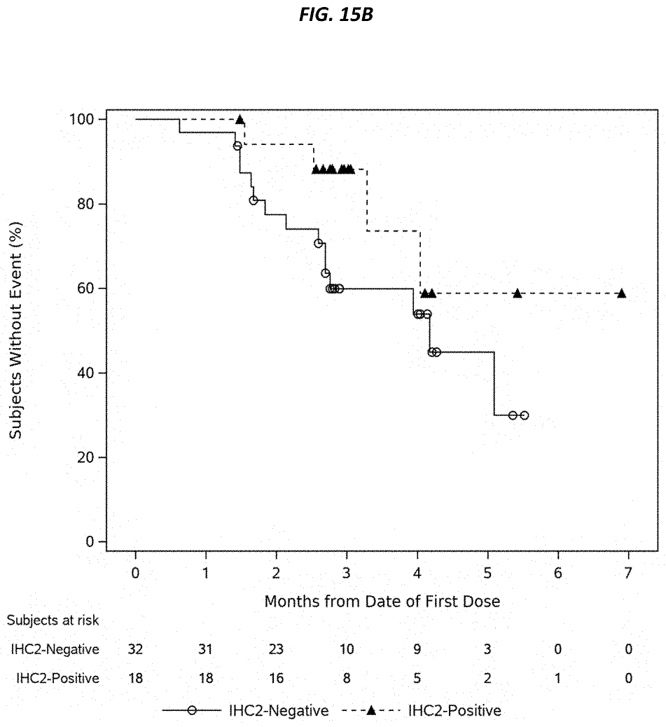

For the analysis, Partial Responders (PR) and Unconfirmed Partial Responders (uPR) were considered “Responders”; and the Progressive Disease, Stable Disease, and not-evaluable/unknown were considered “non-Responders”. Both IHC1 and IHC2 positive groups enriched for Partial Responders (PR) and unconfirmed Partial Responders (uPR) relative to the unselected population. When treating uPR as a PR, the ORR for the IHC1 and IHC2 positive populations are 45.7% (95% exact CI: 28.8-63.4%) and 55.6% (95% exact CI: 30.8-78.5%), respectively; while the ORR for the IHC1 and IHC2 negative populations are 0% (95% exact CI:0-21.8%) and 18.8% (95% exact CI: 7.21-36.4%), respectively. The ORR for the unselected population of subjects with evaluable IHC results is 32% (95% exact CI: 19.5-46.7%). In support of the ORR, swimlane plots demonstrate an enrichment in best change from baseline (defined by RECIST v1.1) of target lesion size by at least a 30% decrease in both IHC positive groups (FIGS. 13A-14B). Sum of target lesion diameters (SoD) was measured as described in E. A. Eisenhauer et al., New response evaluation criteria in solid tumours: Revised RECIST guideline (version 1.1); European J of Cancer 45 (2009) 228-247. At 3 months, both IHC positive groups show better PFS survival relative to their respective negative groups (Table 5, FIGS. 15A-15B).

TABLE 5
Summary of Progression-free Survival by IHC classification; Response
Evaluable at Recommended Phase 2 Combination Dose (RP2CD) Analysis Set in
Combination Therapy (amivantamab + lazertinib)

| | Positive | Negative | Positive | Negative |
|---|---|---|---|---|
| | IHC1 | IHC1 | IHC2 | IHC2 |
| Analysis set: Response Evaluable at | 35 | 15 | 18 | 32 |
| RP2CD Analysis Set in | | | | |
| Combination Therapy | | | | |
| (amivantamab + lazertinib) | | | | |
| Event | 8 (22.9%) | 11 (73.3%) | 4 (22.2%) | 15 (46.9%) |
| Censored | 27 (77.1%) | 4 (26.7%) | 14 (77.8%) | 17(53.1%) |
| Progression-Free Survival (months) | | | | |
| 25th percentile (95% CI) | 3.94 (1.64, | 1.68 (1.41, | 3.29(1.54, | 2.14(1.48, |
| | NE) | 2.69) | NE) | 2.76) |
| Median (95% CI) | NE (3.94, | 2.69 (1.48, | NE (3.29, | 4.17(2.69, |
| | NE) | NE) | NE) | NE) |
| 75th percentile (95% CI) | NE (NE, NE) | 4.17(2.69, | NE (4.04, | NE (4.17, |
| | | NE) | NE) | NE) |
| Range | (0.6, 6.9+) | (1.4, 5.1) | (1.5+, 6.9+) | (0.6, 5.5+) |
| 3-month event free rate (95% CI) | 0.85 (0.67, | 0.40 (0.16, | 0.88 (0.61, | 0.60 (0.40, |
| | 0.93) | 0.63) | 0.97) | 0.75) |
| 6-month event free rate (95% CI) | 0.65 (0.38, | 0 (NE, NE) | 0.59 (0.20, | NE (NE, NE) |
| | 0.82) | | 0.84) | |

RP2CD: amivantamab 1050 mg if baseline weight <80 kg and amivantamab 1400 mg if baseline weight >= 80 kg plus lazertinib 240 mg.
Note:
Response evaluable defined as participants who receive at least one dose of study intervention and who have at least one post baseline disease assessment, clinical progression, or died due to disease progression before the first post baseline disease assessment in addition to having biomarker data available.
IHC1-positive: ≥25% cells are positive for EGFR at 3+ intensity.
IHC1-negative: <25% are positive for EGFR at 3+ intensity.
IHC2-positive: ≥25% of cells are positive for MET at 3+ intensity.
IHC2-negative: <25% of cells are positive for MET at 3+ intensity.
Note:
The response evaluable subjects from first treated 73 subjects are included.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising
a) determining the presence of one or more mutations in tumor DNA obtained from the subject, wherein the one or more mutations are selected from mutations in one or more genes from the RAS/RAF/MEK pathway and mutations in PIK3CA; and
b) (i) identifying the cancer in the subject as susceptible to the treatment with the combination therapy, when tumor DNA from said subject has no said mutations, or (ii) identifying the cancer in the subject as not susceptible to the treatment with the combination therapy, when tumor DNA from said subject has one or more said mutations.

2. A method for treating a cancer in a subject in need thereof, said method comprising
a) determining the presence of one or more mutations in tumor DNA obtained from the subject, wherein the one or more mutations are selected from mutations in one or more genes from the RAS/RAF/MEK pathway and PIK3CA; and
b) (i) when tumor DNA from said subject has no said mutations, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI), or (ii) when tumor DNA from said subject has one or more said mutations, administering to the subject a cancer therapy which does not include the combination therapy used in (i).

3. The method of claim 1 or claim 2, wherein the one or more genes from RAS/RAF/MEK pathway are FGFR3, KRAS, BRAF, ERBB2, ALK, NRAS, PDGFRA and/or RET.

4. The method of claim 3, wherein the mutations in one or more genes from RAS/RAF/MEK pathway comprises FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions.

5. The method of claim 4, wherein the KRAS G12X mutations are KRAS G12D, KRAS G12A, KRAS G12C, and KRAS G12V.

6. The method of claim 3, wherein the mutations in PIK3CA comprise PIK3CA E545K.

7. The method of claim 3, wherein the one or more mutations are further selected from mutations in one or more genes from the WNT/b-catenin pathway.

8. The method of claim 7, wherein the one or more genes from WNT/b-catenin pathway are APC and CTNNB1.

9. The method of claim 8, wherein the mutations in one or more genes from WNT/b-catenin pathway comprise APC Q1469, APC R405, APC S713, CTNNB1 S33P, CTNNB1 S37C, CTNNB1 S37F, and CTNNB1 S45P.

10. The method of claim 1 or claim 2, wherein the cancer is a lung cancer.

11. The method of claim 10, wherein the lung cancer is a non-small cell lung cancer (NSCLC).

12. The method of claim 1 or claim 2, wherein the cancer in the subject is resistant to treatment with an EGFR TKI which is not the same as the EGFR TKI used in the combination therapy.

13. The method of claim 12, wherein the EGFR TKI to which the cancer is resistant is selected from osimertinib, erlotinib, afatinib, rociletinib, olmutinib, and any combination thereof.

14. The method of claim 13, wherein the EGFR TKI to which the cancer is resistant is osimertinib.

15. The method of claim 1 or claim 2, wherein the subject is chemotherapy naïve.

16. The method of claim 1 or claim 2, wherein the tumor DNA from the subject has at least one EGFR-activating mutation.

17. The method of claim 16, wherein the EGFR-activating mutation is selected from exon 19 deletions, and L858R.

18. The method of claim 1 or claim 2, wherein the tumor DNA is circulating tumor DNA (ctDNA).

19. The method of claim 18, wherein the ctDNA is present in a biological sample isolated from the subject.

20. The method of claim 19, wherein the biological sample is a blood sample or a plasma sample.

21. The method of claim 18, wherein ctDNA is isolated from the biological sample prior to mutation identification.

22. The method of claim 16, wherein the tumor DNA is present in a tumor sample isolated from the subject.

23. The method of claim 22, wherein the tumor DNA is isolated from the tumor sample prior to mutation identification.

24. The method of claim 1 or claim 2, wherein the one or more mutations are determined by sequencing.

25. The method of claim 24, wherein the one or more mutations are determined using next-generation sequencing (NGS).

26. The method of claim 1 or claim 2, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

27. The method of claim 26, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

28. The method of claim 26, wherein the bispecific anti-EGFR/c-Met antibody is an IgG1 isotype.

29. The method of claim 26, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

30. The method of claim 26, wherein the bispecific anti-EGFR/c-Met antibody comprises a biantennary glycan structure with a fucose content of about between 1% to about 15%.

31. The method of claim 1 or claim 2, wherein the bispecific anti-EGFR/c-Met antibody is administered intravenously to the subject.

32. The method of claim 31, wherein the bispecific anti-EGFR/c-Met antibody is administered at a dose of between about 140 mg to about 2240 mg.

33. The method of claim 32, wherein the bispecific anti-EGFR/c-Met antibody is administered at a dose of about 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1575 mg, 1600 mg, 2100 mg, or 2240 mg.

34. The method of claim 33, wherein the bispecific anti-EGFR/c-Met antibody is administered at a dose of 1050 mg if the subject has a body weight of less than 80 kg.

35. The method of claim 33, wherein the bispecific anti-EGFR/c-Met antibody is administered at a dose of 1400 mg if the subject has a body weight of greater than or equal to 80 kg.

36. The method of claim 1 or claim 2, wherein the bispecific anti-EGFR/c-Met antibody is administered subcutaneously or intradermally to the subject.

37. The method of claim 36, wherein the bispecific anti-EGFR/c-Met antibody is administered subcutaneously or intradermally at a dose sufficient to achieve a therapeutic effect in the subject.

38. The method of claim 1 or claim 2, wherein the bispecific anti-EGFR/c-Met antibody is administered twice a week, once a week, once in two weeks, once in three weeks or once in four weeks.

39. The method of claim 1 or claim 2, wherein the EGFR TKI administered in combination with the bispecific anti-EGFR/c-Met antibody is lazertinib.

40. The method of claim 1 or claim 2, wherein the EGFR TKI administered in combination with the bispecific anti-EGFR/c-Met antibody is administered at a dose of between about 20 to about 320 mg.

41. The method of claim 40, wherein the EGFR TKI administered in combination with the bispecific anti-EGFR/c-Met antibody is administered at a dose of about 240 mg.

42. The method of claim 40, wherein the EGFR TKI administered in combination with the bispecific anti-EGFR/c-Met antibody is administered daily, every other day, twice a week, or once a week.

43. The method of claim 42, wherein the EGFR TKI administered in combination with the bispecific anti-EGFR/c-Met antibody is administered daily.

44. The method of claim 42, wherein the EGFR TKI administered in combination with the bispecific anti-EGFR/c-Met antibody is administered orally.

45. The method of claim 2, wherein the cancer therapy which does not include the combination therapy used in (i) is a platinum-based chemotherapy.

46. The method of claim 45, wherein the platinum-based chemotherapy comprises carboplatin and/or cisplatin.

47. The method of claim 1 or claim 2, comprising obtaining a biological sample from the subject prior to step (a), wherein said biological sample comprises tumor DNA, and optionally purifying said tumor DNA from said biological sample.

48. A method for determining whether a cancer in a subject is susceptible to a treatment with a combination therapy comprising a bispecific anti-epidermal growth factor receptor (EGFR)/hepatocyte growth factor receptor (c-Met) bispecific antibody and an EGFR tyrosine kinase inhibitor (TKI), said method comprising
a) determining expression level of EGFR or MET in a tumor sample obtained from the subject using immunohistochemistry (IHC),
b) determining a staining intensity score, on a scale from 0 to 3+, based on the expression level of EGFR or MET determined in step (a), and
c) (i) identifying the cancer in the subject as susceptible to the treatment with the combination therapy, when the staining intensity score is 3+, or (ii) identifying the cancer in the subject as not susceptible to the treatment with the combination therapy, when the staining intensity score is less than 3+.

49. The method of claim 48, wherein step (c) comprises identifying the cancer in the subject as susceptible to the treatment with the combination therapy, when the staining intensity score is 3+ in greater than or equal to 25% of the cells of the tumor sample, or (ii) identifying the cancer in the subject as not susceptible to the treatment with the combination therapy, when the staining intensity score is 3+ in less than 25% cells of the tumor sample.

50. A method for treating a cancer in a subject in need thereof, said method comprising
a) determining the expression level of EGFR or MET in a tumor sample obtained from the subject using immunohistochemistry (IHC),
b) determining a staining intensity score, on a scale from 0 to 3+, based on the expression level of EGFR or MET determined in step (a), and
c) (i) when the staining intensity score is 3+, administering to the subject a therapeutically effective amount of a combination therapy comprising a bispecific anti-epidermal growth factor receptor (EGFR)/hepatocyte growth factor receptor (c-Met) bispecific antibody and an EGFR tyrosine kinase inhibitor (TKI); or (ii) when the staining intensity score is less than 3+, not administering to the subject the combination therapy used in (i) or administering to the subject a cancer therapy which does not include the combination therapy used in (i).

51. The method of claim 50, wherein step (c) comprises (i) when the staining intensity score is 3+ in greater than or equal to 25% of the cells of the tumor sample, administering to the subject a therapeutically effective amount of the combination therapy; or (ii) when the staining intensity score is 3+ in less than 25% cells of the tumor sample, not administering to the subject the combination therapy used in (i) or administering to the subject a cancer therapy which does not include the combination therapy used in (i).

52. The method of claim 48 or claim 50, wherein the cancer is a lung cancer.

53. The method of claim 52, wherein the lung cancer is a non-small cell lung cancer (NSCLC).

54. The method of claim 48 or claim 50, wherein the cancer in the subject is resistant to treatment with an EGFR TKI which is not the same as the EGFR TKI used in the combination therapy.

55. The method of claim 54, wherein the EGFR TKI to which the cancer is resistant is selected from osimertinib, erlotinib, afatinib, rociletinib, olmutinib, and any combination thereof.

56. The method of claim 55, wherein the EGFR TKI to which the cancer is resistant is osimertinib.

57. The method of claim 48 or claim 50, wherein the subject is chemotherapy naïve.

58. The method of claim 48 or claim 50, wherein the tumor of the subject has at least one EGFR-activating mutation.

59. The method of claim 58, wherein the EGFR-activating mutation is selected from exon 19 deletions, and L858R.

60. The method of claim 48 or claim 50, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

61. The method of claim 60, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

62. The method of claim 60, wherein the bispecific anti-EGFR/c-Met antibody is an IgG1 isotype.

63. The method of claim 60, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

64. The method of claim 60, wherein the bispecific anti-EGFR/c-Met antibody comprises a biantennary glycan structure with a fucose content of about between 1% to about 15%.

65. The method of claim 60, wherein the bispecific anti-EGFR/c-Met antibody is administered intravenously to the subject.

66. The method of claim 65, wherein the bispecific anti-EGFR/c-Met antibody is administered at a dose of between about 140 mg to about 2240 mg.

67. The method of claim 66, wherein the bispecific anti-EGFR/c-Met antibody is administered at a dose of about 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1575 mg, 1600 mg, 2100 mg, or 2240 mg.

68. The method of claim 67, wherein the bispecific anti-EGFR/c-Met antibody is administered at a dose of 1050 mg if the subject has a body weight of less than 80 kg.

69. The method of claim 67, wherein the bispecific anti-EGFR/c-Met antibody is administered at a dose of 1400 mg if the subject has a body weight of greater than or equal to 80 kg.

70. The method of claim 60, wherein the bispecific anti-EGFR/c-Met antibody is administered subcutaneously or intradermally to the subject.

71. The method of claim 70, wherein the bispecific anti-EGFR/c-Met antibody is administered subcutaneously or intradermally at a dose sufficient to achieve a therapeutic effect in the subject.

72. The method of claim 60, wherein the bispecific anti-EGFR/c-Met antibody is administered twice a week, once a week, once in two weeks, once in three weeks or once in four weeks.

73. The method of claim 60, wherein the EGFR TKI administered in combination with the bispecific anti-EGFR/c-Met antibody is lazertinib.

74. The method of claim 60, wherein the EGFR TKI administered in combination with the bispecific anti-EGFR/c-Met antibody is administered at a dose of between about 20 to about 320 mg.

75. The method of claim 74, wherein the EGFR TKI administered in combination with the bispecific anti-EGFR/c-Met antibody is administered at a dose of about 240 mg.

76. The method of claim 60, wherein the EGFR TKI administered in combination with the bispecific anti-EGFR/c-Met antibody is administered daily, every other day, twice a week, or once a week.

77. The method of claim 76, wherein the EGFR TKI administered in combination with the bispecific anti-EGFR/c-Met antibody is administered daily.

78. The method of claim 60, wherein the EGFR TKI administered in combination with the bispecific anti-EGFR/c-Met antibody is administered orally.

79. The method of claim 50, wherein the cancer therapy which does not include the combination therapy used in (i) is a platinum-based chemotherapy.

80. The method of claim 79, wherein the platinum-based chemotherapy comprises carboplatin and/or cisplatin.

81. The method of claim 48 or claim 50, comprising obtaining a tumor sample from the subject prior to step (a).

82. A diagnostic kit comprising (i) one or more reagents for determining the presence of one or more mutations in tumor DNA from a subject with a cancer, and (ii) optionally packaging and/or instructions for use, wherein the one or more mutations are selected from mutations in one or more genes from the RAS/RAF/MEK pathway and mutations in PIK3CA.

83. The diagnostic kit of claim 82, wherein the one or more genes from RAS/RAF/MEK pathway are FGFR3, KRAS, BRAF, ERBB2, ALK, NRAS, PDGFRA and/or RET.

84. The diagnostic kit of claim 83, wherein the mutations in one or more genes from RAS/RAF/MEK pathway comprise FGFR3 fusions, BRAF G469A, BRAF V600E, ERBB2 copy number alterations, ALK fusions, ERBB2 I767M, ERBB2 V777L, KRAS A18V, KRAS copy number alterations, KRAS G12X (X being any amino acid), NRAS Q61R, PDGFRA copy number alterations, and RET fusions.

85. The diagnostic kit of claim 84, wherein the KRAS G12X mutations are KRAS G12D, KRAS G12A, KRAS G12C, and KRAS G12V.

86. The diagnostic kit of claim 82, wherein the mutations in PIK3CA comprise PIK3CA E545K.

87. The diagnostic kit of claim 82, wherein the one or more mutations are further selected from mutations in one or more genes from the WNT/b-catenin pathway.

88. The diagnostic kit of claim 87, wherein the one or more genes from WNT/b-catenin pathway are APC and CTNNB1.

89. The diagnostic kit of claim 88, wherein the mutations in one or more genes from WNT/b-catenin pathway comprise APC Q1469, APC R405, APC S713, CTNNB1 S33P, CTNNB1 S37C, CTNNB1 S37F, and CTNNB1 S45P.

90. The diagnostic kit of claim 82, wherein the tumor DNA is circulating tumor DNA (ctDNA).

91. The diagnostic kit of claim 90, wherein the ctDNA is present in a biological sample isolated from the subject.

92. The diagnostic kit of claim 91, wherein the biological sample is a blood sample or a plasma sample.

93. The diagnostic kit of claim 82, wherein the tumor DNA is present in a tumor sample isolated from the subject.

94. The diagnostic kit of claim 91, further comprising one or more reagents for purifying said tumor DNA from said biological sample from the subject.

95. The diagnostic kit of claim 82, wherein the one or more reagents can be used with a sequencing technique to determine the one or more mutations.

96. The diagnostic kit of claim 82, wherein the one or more reagents can be used with next-generation sequencing (NGS) to determine the one or more mutations.

97. A diagnostic kit comprising (i) one or more reagents for determining the expression level of EGFR and/or MET in a tumor sample from a subject with a cancer, and (ii) optionally packaging and/or instructions for use.

98. The diagnostic kit of claim 97, wherein the one or more reagents can be used with immunohistochemistry (IHC) to determine the expression level of EGFR and/or MET.
